(19) United States
(12) Patent Application Publication
(10) Pub. No.: US 2005/0049712 A1
(43) Pub. Date: Mar. 3, 2005

(54) Shoulder Positioning fixture, trials and associated method

(76) Inventors: Jeffrey M. Ondrla, Leesburg, IN (US); Andrea L. Hahn, Petersburg, MI (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/650,029

(22) Filed: Aug. 27, 2003

(51) Int. Cl. A61F 2/46; A61F 2/36; A61B 17/88

(52) U.S. Cl. 623/22.12; 606/86; 623/23.31; 623/23.44

(57) ABSTRACT

A positioning fixture for positioning at least one of a trial and a prosthesis in the medullary canal of a long bone is provided. The fixture includes a body and a member for cooperation with the body. The member extends from the body through at least a portion of the long bone and into the medullary canal. The member is adapted to support at least one of the trial and the prosthesis as the one of the trial and the prosthesis is positioned with respect to the long bone.

### TECHNICAL FIELD OF THE INVENTION

The present invention relates generally to the field of orthopaedics, and more particularly, to an instrument for use in arthroplasty.

### BACKGROUND OF THE INVENTION

The present invention relates to implantable articles and methods of for implanting such articles. More particularly the invention relates to a bone prosthesis, prosthesis trial, instrument and method for implanting the same.

There are known to exist many designs for and methods for implanting implantable articles, such as bone prostheses. Such bone prostheses include components of artificial joints, such as elbows, hips, knees and shoulders.

One such implantable prosthesis is a shoulder prosthesis. During the life time of a patient, it may be necessary to perform a total shoulder replacement procedure on a patient as a result of, for example, disease or trauma, for example disease from osteoarthritis or rheumatoid arthritis. Currently, most implantable shoulder prostheses are total shoulder prostheses. In a total shoulder replacement procedure, a humeral component having a head portion is utilized to replace the natural head portion of the upper arm bone or humerus. The humeral component typically has an elongated intermedullary stem, which is utilized to secure the humeral component to the patient's humerus. In such a total shoulder replacement procedure, the natural glenoid surface of the scapula is resurfaced or otherwise replaced with a glenoid component that provides a bearing surface for the head portion of the humeral component.

When constructing damaged or diseased joints with artificial prostheses, it is desirable to position the component of the prosthesis such that the structure and function of a properly functioning natural joint is replicated to the greatest degree possible. This involves consideration of several factors. For example, the components of the prosthesis must be inserted such that the patient's joint has the desired height and version after the surgery. It is also desirable that the range of motion of the joint postoperatively is, to the extent possible, the same as that of a healthy joint. Some of the factors relative to proper placement include insertion depth of the prosthesis component relative to the bone and rotational orientation of the prosthesis components.

When reconstructing a diseased joint, the proper positioning of the replacement shoulder prosthesis stem is determined by reference to the resected surface of the humerus. For such surgeries bony landmarks or fixturing is utilized to perform an accurate resection. The accurate resection thus will place the prosthetic stem in the proper position for functioning.

However, when reconstructing damaged joints when the proximal position of the proximal humerus is severely damaged, the positioning of the prosthetic stem in the humerus is much more difficult. Prior art attempts at properly positioning a humeral stem have consisted of packing material such as gauze around the prosthetic stem in a first position and performing a trial reduction to determine the appropriateness of the position and adjusting that position based upon that trial. Such a process is time consuming at best and may often lead to suboptimal positioning of the prosthesis.

Attempts have been made to provide for an accurate way of positioning a humeral stem in a humerus based upon a damaged or trauma need for surgery. These devices have been large and cumbersome and because of their size have not permitted a full range of motion during the trial.

The present invention attempts to overcome at least some of the aforementioned problems.

### SUMMARY OF THE INVENTION

Present invention provides for a positioning fixture or jig which holds a trial shoulder prosthesis in place during proximal humeral fracture surgery. The positioning jig provides for support for the prosthesis by including members which are inserted through the bone adjacent the positioning fixture. A construction in which the fixture is connected to the prosthesis directly through the bone provides for a small size and simple construction in comparison to prior art positioning fixtures in which the fixture must extend to the resected portion of the prosthesis where the fixture engages the proximal portion of the trial. The small size of the positioning fixture of the present invention allows for a more complete trial reduction of the shoulder without impinging from the positioning jig. The fixturing jig of the present invention allows for the proper height and version of the implant to be determined.

According to one embodiment of the present invention, there is provided a positioning fixture for positioning at least one of a trial and a prosthesis in the medullary canal of a long bone. The fixture includes a body and a member for cooperation with the body. The member extends from the body through at least a portion of the long bone and into the medullary canal. The member is adapted to support at least one of the trial and the prosthesis as the one of the trial and the prosthesis is positioned with respect to the long bone.

According to another embodiment of the present invention there is provided a positioning fixture for positioning at least one of a trial and a prosthesis in the medullary canal of a long bone. The fixture includes a body and a member for cooperation with the body. The member extends from the body through at least a portion of the long bone and into the medullary canal. The member is adapted to permit relative motion between the body and the one of least one of the trial and the prosthesis as the one of the trial and the prosthesis cooperates with the member.

According to still another embodiment of the present invention there is provided a kit for use in performing joint arthroplasty. The kit includes one of a trial and a prosthesis. The one of the trial and the prosthesis includes a cooperation feature and a positioning fixture. The positioning fixture is used for positioning at least one of a trial and a prosthesis in the medullary canal of a long bone. The fixture includes a body and a member for cooperation with the body. The member extends from the body through at least a portion of the long bone and into the medullary canal. The member is adapted to support at least one of the trial and the prosthesis as the one of the trial and the prosthesis is positioned with respect to the long bone.

According to yet another embodiment of the present invention there is provided a prosthesis for use with a positioning fixture for positioning the prosthesis in the medullary canal of a long bone. The prosthesis includes a cooperation feature. The prosthesis is adapted to be supported at the cooperation feature by the positioning fixture as the prosthesis is positioned with respect to the long bone.

According to yet another embodiment of the present invention there is provided a trial for use with a positioning fixture for positioning the trial in the medullary canal of a long bone. The trial includes a cooperation feature. The trial is adapted to be supported at the cooperation feature by the positioning fixture as the trial is positioned with respect to the long bone.

According to a further embodiment of the present invention, there is provided a method for providing joint arthroplasty. The method includes the step of providing a kit including a prosthesis including a cooperation feature and a positioning fixture for positioning the prosthesis in the medullary canal of a long bone. The fixture includes a body and a member for cooperation with the body. The member extends from the body through at least a portion of the long bone and into the medullary canal. The method also includes the steps of cutting an incision in the patient, preparing the medullary cavity, positioning the positioning fixture with respect to the bone, assembling the prosthesis onto the medullary cavity and the fixture, performing a reduction of the prosthesis, adjusting the position of the prosthesis and performing additional trials as necessary, and implanting the prosthesis in the medullary cavity.

The technical advantages of the present invention include the ability to provide a more complete trial reduction. Utilizing the present invention, the alignment fixture of the present invention has a very low profile or small shape and, therefore, provides little impingement or interference with the motion of the arm through the trial reduction. For example, according to one aspect of the present invention, the fixture contacts the trial through holes in the exterior cortical wall of the bone. Thus, the present invention provides for a small simple fixture to support the trial directly through the bone rather than to provide for support of the prosthesis to the fixture through the resected end of the bone. Thus, the present invention provides for a more complete trial reduction.

Another technical advantage of the present invention includes the ability of the fixture to remain attached to the bone when the prosthesis is adjusted for version and height. For example, according to one aspect of the present invention, a feature is placed on the periphery of the stem of the trial to cooperate with a feature of the fixture that extends through the cortical wall of the bone. Thus, the present invention provides for the ability to adjust the prosthesis for version and height relative to the bone while the fixture remains attached to the bone.

Yet another technical advantage of the present invention includes the ability of the fixture of the present invention to provide a simple fixation to the bone. For example, according to one aspect of the present invention, k wires are slideably fitted through the fixture and are secured directly to the bone. Thus, the present invention provides for a simple fixation to the bone. The k wires can be quickly drilled and left in position in the bone until the locking screws are engaged. Preferably the K-wires are removed prior to trial reduction to assist in the insertion of the stem and to optimize the range of motion.

Another technical advantage of the present invention includes a fixation to the bone that is simple, non-invasive to the bone and easy to remove. For example, according to one aspect of the present invention a clamp in the form of, for example, a set screw is threadably secured to the fixture and is directly in contact with the periphery of the bone. Thus, the present invention provides for a simple, noninvasive fixation to the bone.

A further technical advantage of the present invention includes the simple, inexpensive and easy to use nature of the fixture of the present invention. For example, according to one aspect of the present invention, the fixture is in the form of a ring with pins to pass through the cortical wall of the bone and to directly engage in grooves in the distal portion of the stem of the trial. The fixture, thus, is small, in the form of a ring and pins, inexpensive, in the form of a simple, inexpensive ring and a simple, inexpensive pin and is easy to use. It slides over the bone and the pins are engaged directly into the bone.

Yet another technical advantage of the present invention includes the use of a simple guide for drilling holes in the long bone. For example, according to one aspect of the present invention, a guide in the form of a bushing with a handle extending from the bushing is used to slideably fit in the threaded holes of the ring. This guide provides for simple and time saving guiding of the drill for drilling the holes through the cortical bone of the long bone.

Yet another technical advantage of the present invention includes the ability of the fixture to be used with the implant and to position the implant in the prosthesis during the cementing process. For example, according to one aspect of the present invention, the pins used to position the prosthesis in the bone includes pins that extend from the fixture inwardly toward the prosthesis and that are made of a humanly compatible material or a resorbable material. The pins may be severed from the fixture after the cementing process and remain in the patient. Thus, the present invention provides for the use of a fixture to center the prosthesis during this cementing process.

Other technical advantages of the present invention will be readily apparent to one skilled in the art from the following figures, descriptions and claims.

### BRIEF DESCRIPTION OF THE DRAWINGS

For a more complete understanding of the present invention and the advantages thereof, reference is now made to the following description taken in connection with the accompanying drawings, in which:

FIG. 1 is a plan view of a fixture in accordance with an embodiment of the present invention showing the pins and the K wires in position;

FIG. 2 is a perspective view of the fixture of FIG. 1;

FIG. 3 is a perspective view of the fixture of FIG. 1 showing the pins and the K wires showing the pins and the K wires removed;

FIG. 4 is a plan view of the fixture of FIG. 1 showing the pins in position;

FIG. 5 a plan view of a pin for use in the fixture of FIG. 1;

FIG. 6 is a rear view of the fixture of FIG. 1 showing the pins in position;

FIG. 6A is a plan view of a locking screw for use with the fixture of FIG. 1.

FIG. 6B is a plan view of the fixture of FIG. 1 in position on a trial showing the trial in a first and a second position;

FIG. 7 is a plan view of a humeral stem of a humeral trial for use with the fixture of FIG. 1;

FIG. 8 is a cross sectional view of FIG. 7 along the line 8-8 in the direction of the arrows;

FIG. 8A is a cross sectional view of another humeral stem of a humeral trial for a use with the fixture of FIG. 1;

FIG. 8B is a cross sectional view of another humeral stem of a humeral trial with the fixture of FIG. 1;

FIG. 8C is a cross sectional view of another humeral stem of a humeral trial for use with the fixture of FIG. 1;

FIG. 8D is a cross sectional view of another humeral stem of a humeral trial with a smooth periphery for use with the fixture of FIG. 1;

FIG. 9 is a cross sectional view of an alternate construction of a humeral stem for use with the humeral stem of FIG. 7;

FIG. 10 is a plan view of a humeral stem of a humeral prosthesis for use with the fixture of FIG. 1;

FIG. 11 is a cross sectional view of FIG. 10 along the line 11-11 in the direction of the arrows;

FIG. 11A is a cross sectional view of another humeral stem of a humeral prosthesis for of use with the fixture of FIG. 1;

FIG. 11B is a cross sectional view of another humeral stem of a humeral prosthesis for use with the fixture of FIG. 1;

FIG. 11C is a cross sectional view of another humeral stem of a humeral prosthesis for use with the fixture of FIG. 1;

FIG. 11D is a view of another humeral stem of a humeral prosthesis with a smooth periphery for use with the fixture of FIG. 1;

FIG. 12 is a cross sectional view of an alternate construction of a humeral stem for use with the humeral stem of FIG. 10;

FIG. 13 is a plan view partially in cross-section of the humeral stem of FIG. 7 shown in position in the medullary canal of a humerus with the fixture of FIG. 1 shown in position in engagement with the humerus;

FIG. 14 is a plan view in accordance with another embodiment of the fixture of the present invention with a screw for drawing the fixture portions together showing the pins in position;

FIG. 15 is a plan view in accordance with yet another embodiment of the fixture of the present invention with an alignment rod for aligning the fixture to the humerus showing the pins in position;

FIG. 16 is a plan view of a humeral stem of a humeral trial with alignment features over only a portion of the periphery of the stem for use with the fixture of FIG. 15;

FIG. 17 is a cross sectional view of FIG. 16 along the line 17-17 in the direction of the arrows;

FIG. 18 is a plan view of a humeral stem of a humeral prosthesis with alignment features over only a portion of the periphery of the stem for use with the fixture of FIG. 15;

FIG. 19 is a cross sectional view of the stem of FIG. 18 along the line 19-19 in the direction of the arrows;

FIG. 20 is a plan view in accordance with another embodiment of the fixture of the present invention with two relatively moving non-circular portions for securing the fixture to the bone showing the pins in position;

FIG. 21 is a cross sectional view of the fixture of FIG. 20 along the line 21-21 in the direction of the arrows;

FIG. 22 is a perspective view in accordance with a further embodiment of the fixture of the present invention with a plurality of eccentric clamp portions for securing the fixture to the bone showing the pins in position;

FIG. 22A is a perspective view of one of the eccentric clamp portions of the fixture of FIG. 22;

FIG. 23 is a plan view of the fixture of FIG. 22;

FIG. 24 is an enlarged view of an eccentric clamp portion with a pawl lock for use with the fixture of FIG. 22;

FIG. 25 is an enlarged view of an eccentric clamp portion with an external hex head and a socket cap stem for use with the fixture of FIG. 22;

FIG. 26 is an enlarged view of wrench for use in securing the an eccentric clamp portion of FIG. 25;

FIG. 27 is a perspective view in accordance with a further embodiment of the fixture of the present invention with a tapered bore showing the pins in position;

FIG. 28 is a perspective view of a tapered wedge for use in securing the fixture of FIG. 27 to the long bone;

FIG. 29 is a partial plan view of the fixture of FIG. 27 with the tapered wedge of FIG. 28 installed in the fixture;

FIG. 30 is a plan view in accordance with a further embodiment of the fixture of the present invention with a collapseable spring for securing the fixture to the long bone showing the pins in position;

FIG. 31 is a plan view in accordance with a further embodiment of the fixture of the present invention with tapered bored sections showing the pins in position;

FIG. 32 is a perspective view of a tapered wedge for use in securing the fixture of FIG. 31 to the long bone;

FIG. 33 is a perspective view in accordance with a further embodiment of the fixture of the present invention two rings forming a cam locking arrangement showing the pins in position;

FIG. 34 is a cross sectional view of the fixture of FIG. 33;

FIG. 35 is a top view partially in cross section of the fixture of FIG. 33 showing the attachment of the parts of each ring;

FIG. 36 is a plan view of a fixture in accordance with yet another embodiment of the present invention similar to that of FIG. 1 with the pins made of a biocompatible material for use in securing the prosthesis in the long bone medullary canal during cementing;

FIG. 37 is a plan view of a drill guide including a handle for guiding a drill for preparing a hole for receiving the pin of the fixture of the present invention;

FIG. 38 is a rear view of the guide of FIG. 37;

FIG. 39 is a end view of the guide of FIG. 37; and

FIG. 40 is a flow chart of a method for performing shoulder arthroplasty in accordance with another embodiment of the present invention.

### DETAILED DESCRIPTION OF THE INVENTION

Embodiments of the present invention and the advantages thereof are best understood by referring to the following descriptions and drawings, wherein like numerals are used for like and corresponding parts of the drawings.

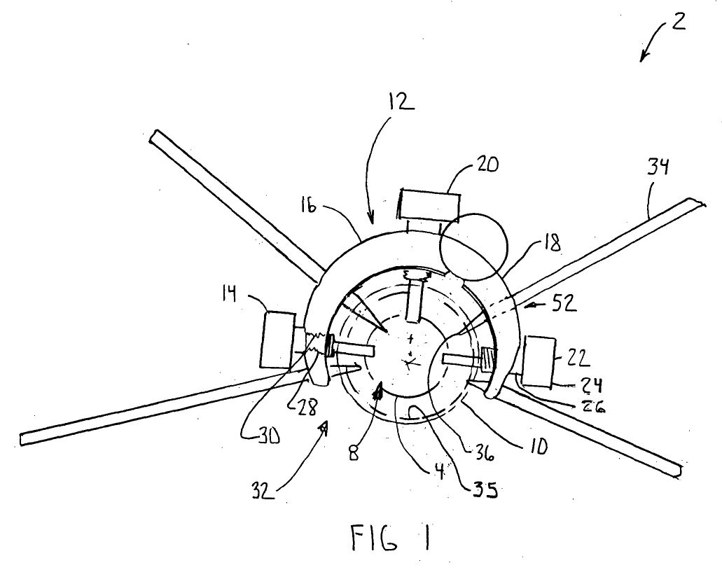

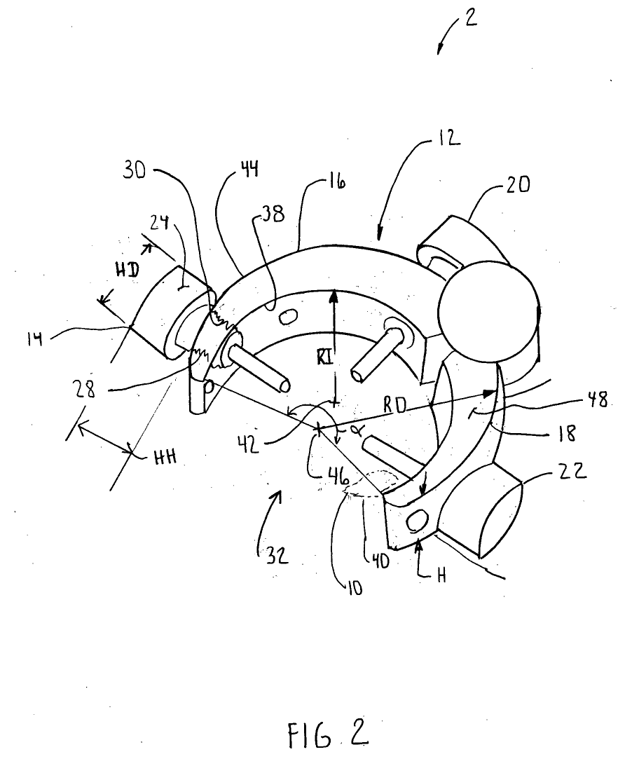

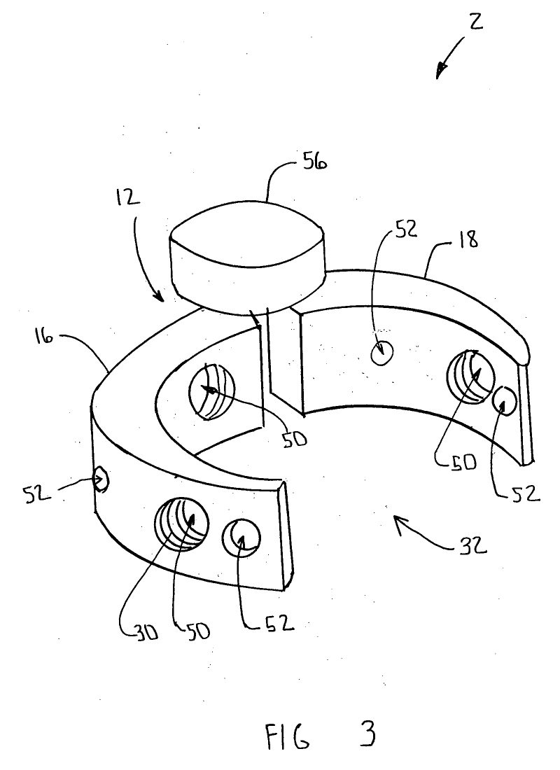

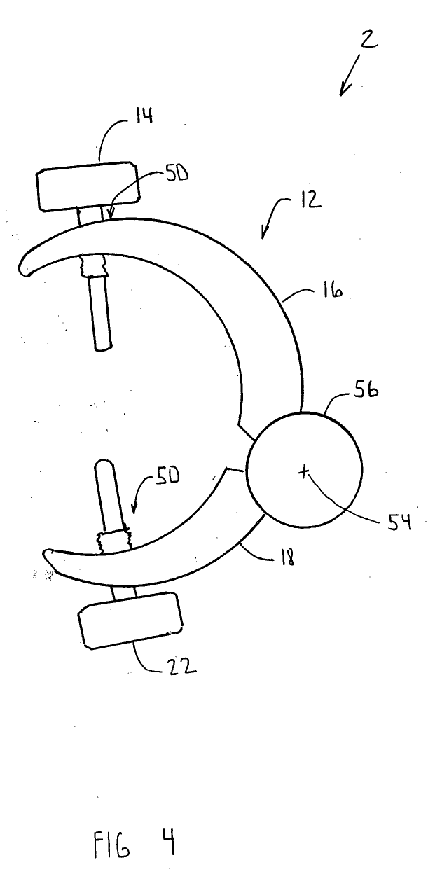

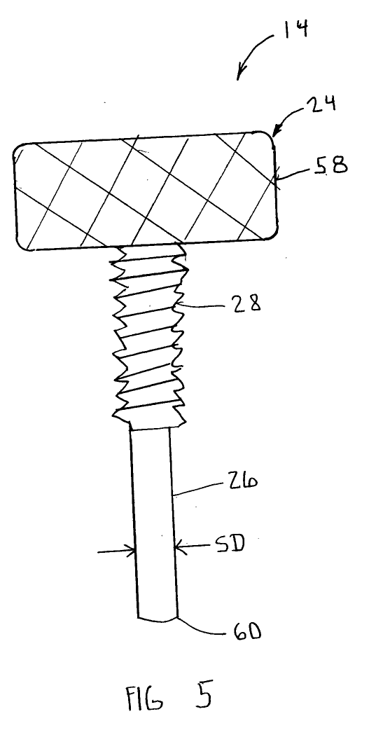

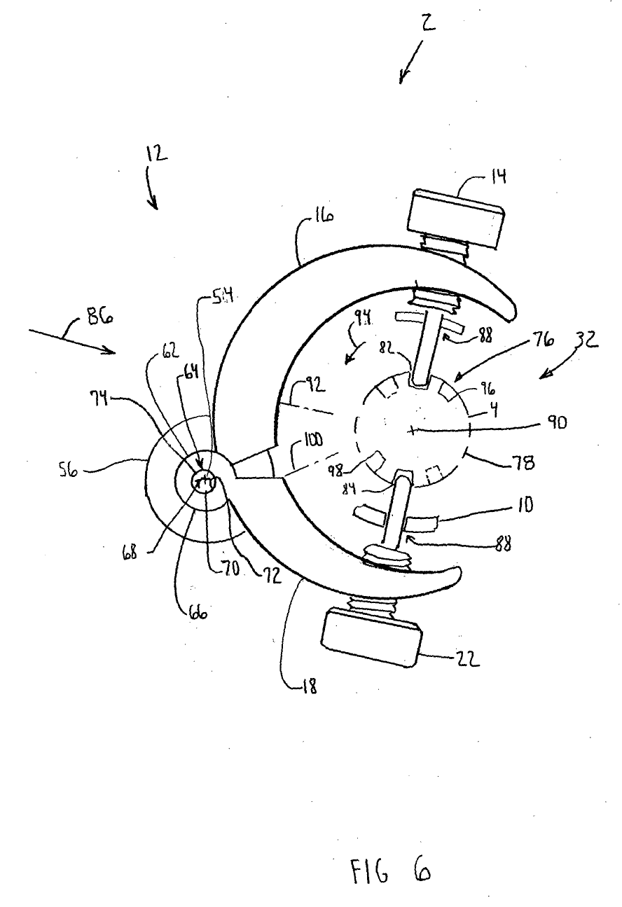

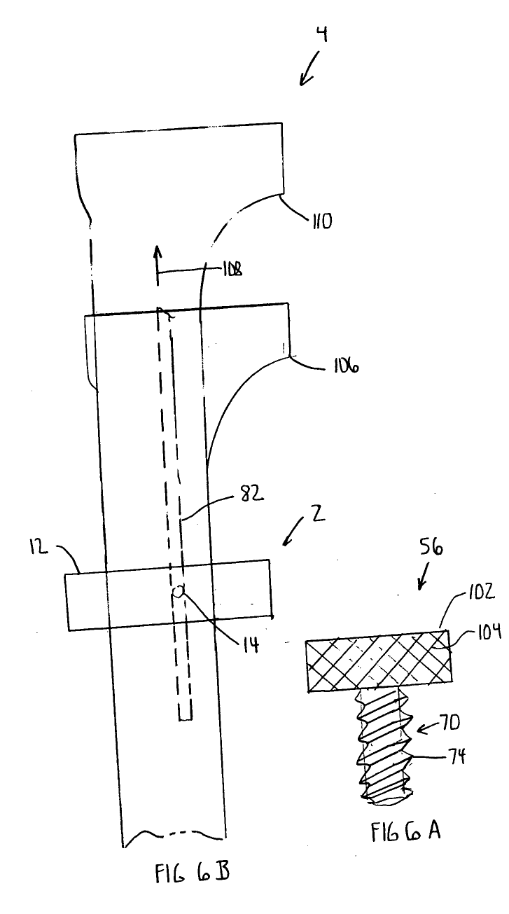

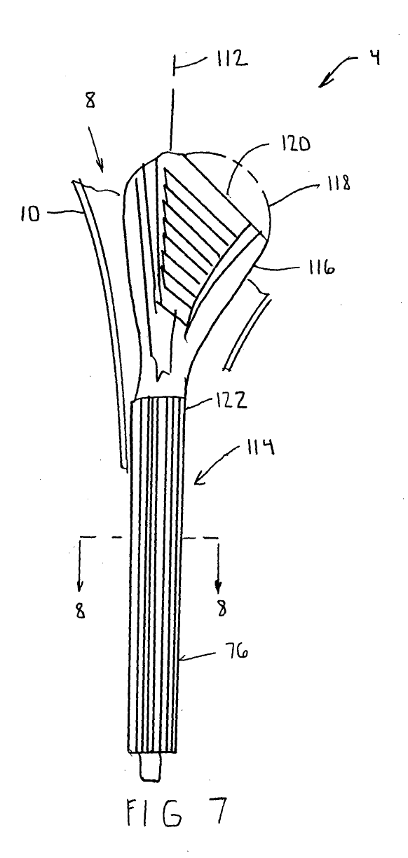

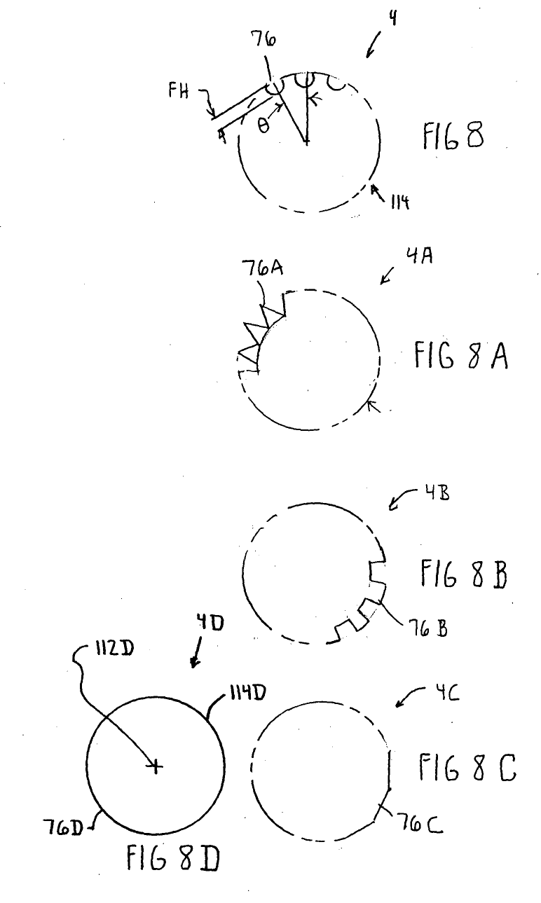

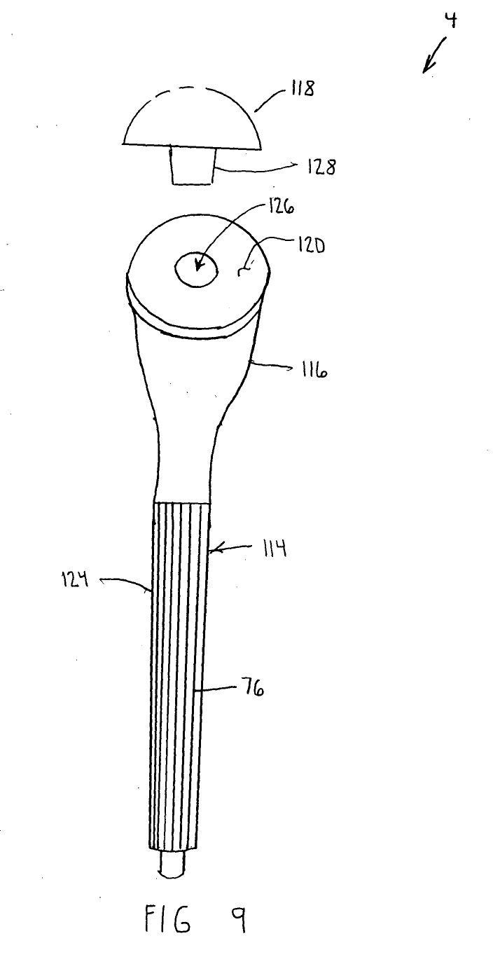

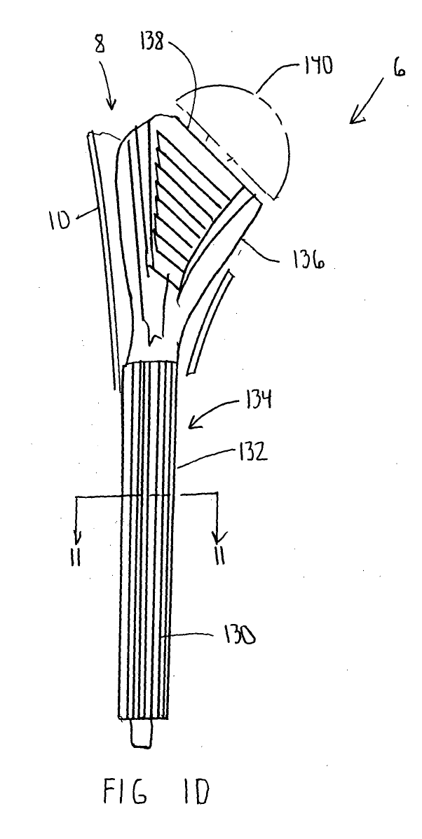

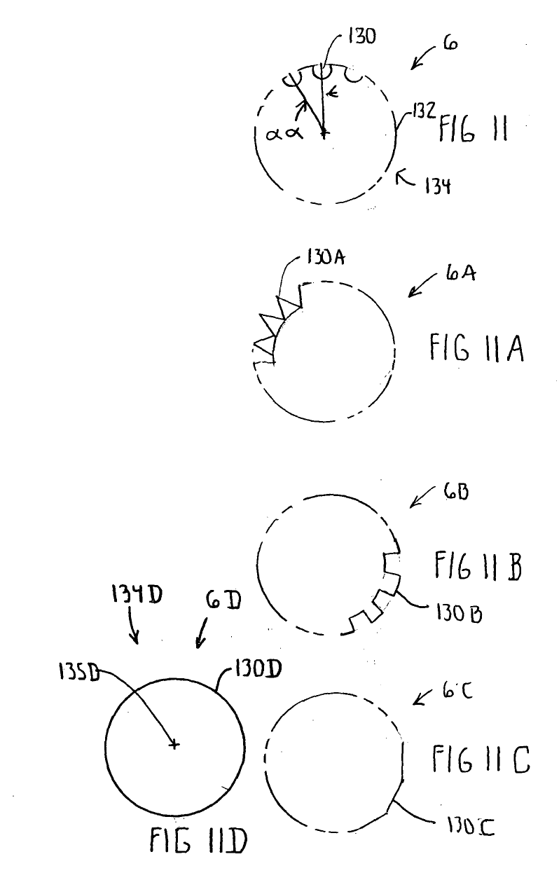

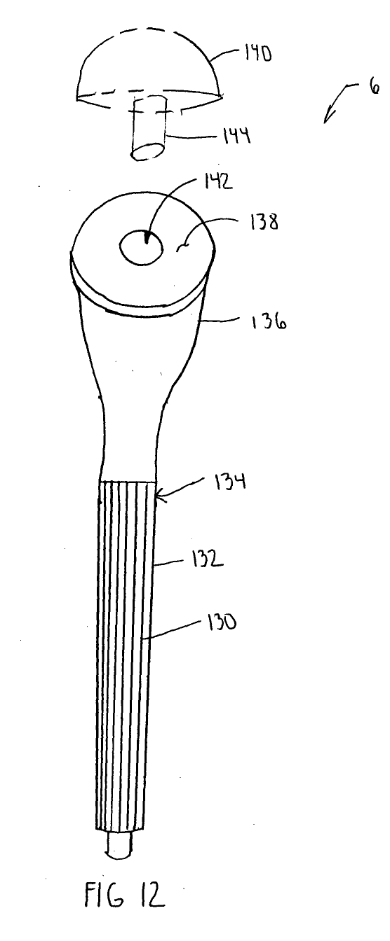

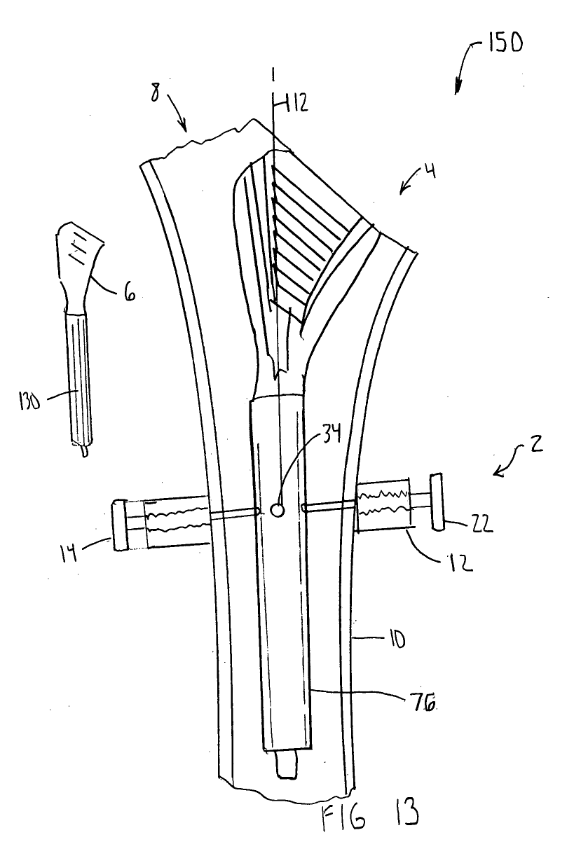

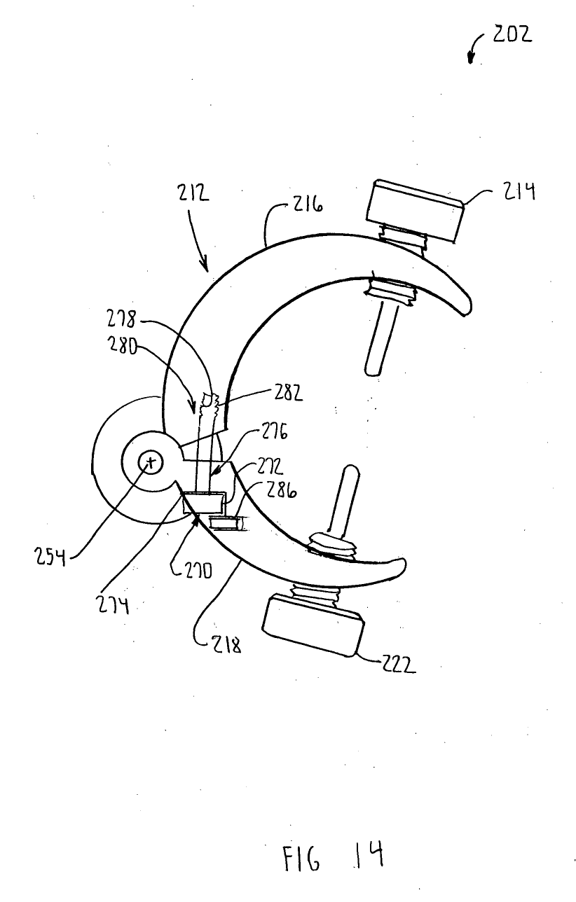

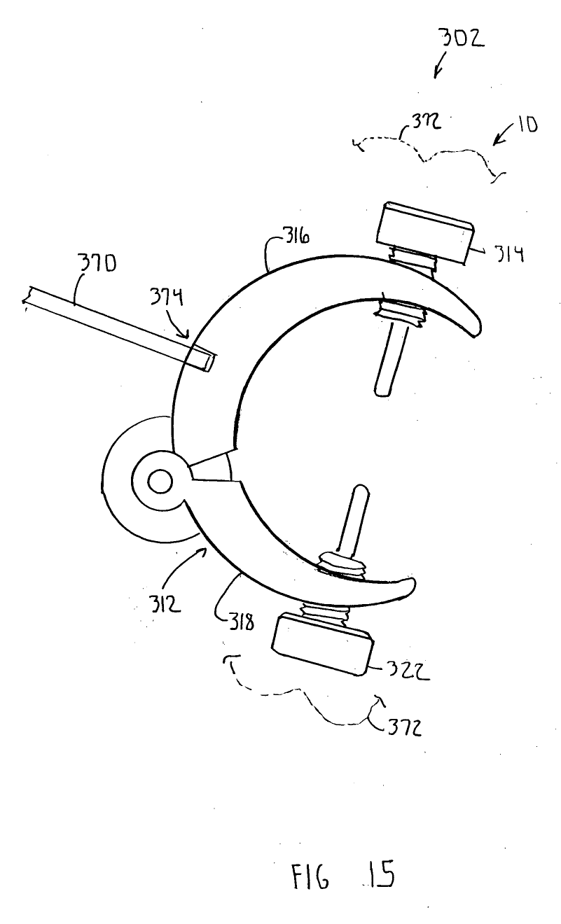

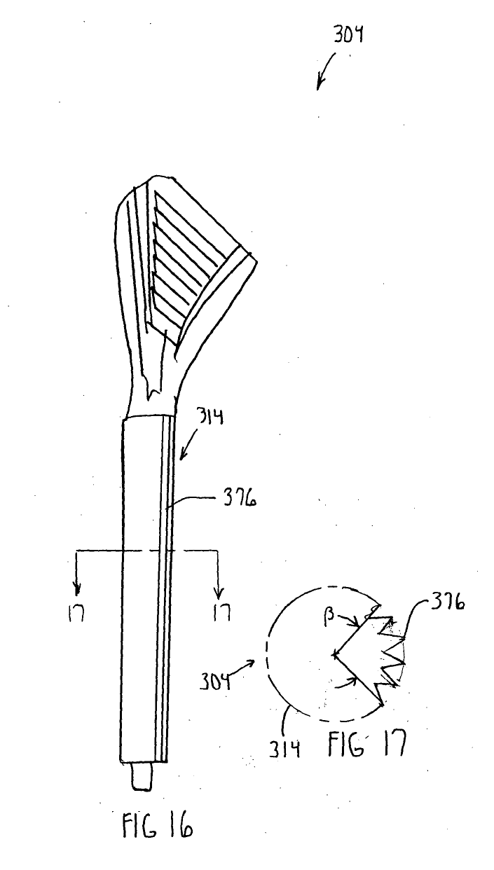

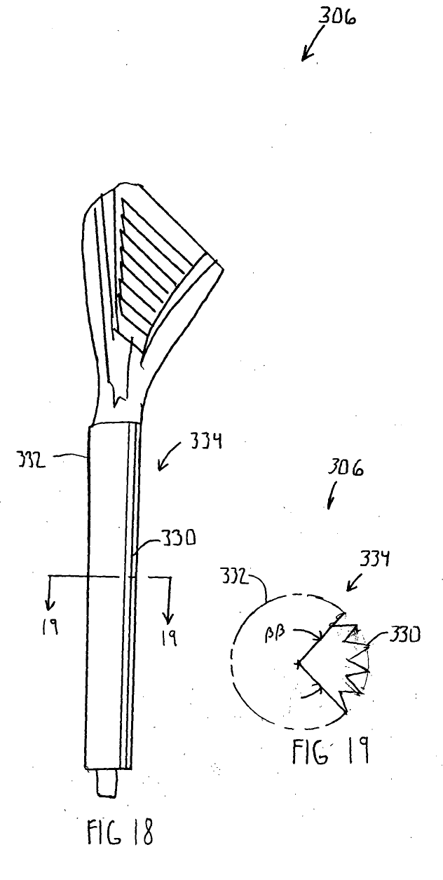

According to the present invention and referring now to FIG. 1, a positioning fixture 2 is shown for positioning either the trial 4 or the prosthesis 6 (see FIG. 10) in the medullary canal 8 of a long bone 10. The positioning fixture 2 includes a body 12 and a member 14 for cooperation with the body 12. The member 14 extends from the body 12 through a portion of the long bone 10 and into the medullary canal 8. The member 14 may support the trial 4 or the prosthesis 6 as the trial 4 or the prosthesis 6 is positioned with respect to the long bone 10.

The member 14 may be adapted to permit relative motion between the body 12 and the either the trial 4 or the prosthesis 6 as the trial 4 or the prosthesis 6 cooperates with the member 14.

The body 12 may have any suitable size and shape capable of cooperating with the member 14 and assisting and providing the support to the trial 4. For example, the body 12 may be in the form of a ring for at least partially surrounding the long bone 10. For example, the ring 12 may include a first portion 16 and a second portion 18 which is moveably attached to the first portion 16. The moveable first portion 16 and moveable second portion 18 may assist in more closely conforming to the long bone 10.

While it should be appreciated that the positioning fixture 2 may include a solitary member 14, the positioning fixture 2 may further include a second member 20. The second member 20 may have a configuration similar to that of the first member 14 and is preferably spaced from the first member 14. The second member 20 assists in providing the support and assists in the positioning of the trial with the respect to the positioning fixture 2 and consequently with the long bone 10.

Similarly, it should be appreciated that in addition to the first member 14 and the second member 20, additional members, for example a third member 22, may be utilized to support the trial and to position the trial relative to the long bone 10. Similarly to the second member 20, the third member 22 is preferably spaced from the first member 14 and the second member 20. The third member 22 may have a shape or configuration similar to that of the first member 14 or the second member 20.

The first member 14 may have any suitable size and shape capable of cooperating with the body 12 and for supporting the trial 4. For example, and as shown in FIG. 1, the first member 14 may be in the form of a pin. The pin 14 may include a head 24 and a stem 26 extending from the stem 24. External threads 28 may be formed on the stem 26 for cooperating with internal threads 30 on the body 12. Conversely, the stem 26 of the pin may be slideably attached to the body 12.

The body 12 may have any suitable shape capable of providing a support around the periphery of the long bone 10. For example, the body 12 may have an opening 32 such that the body 12 has a generally U shaped cross section. For example, the body 12 may have a crescent shaped cross section.

While it should be appreciated that the positioning fixture 2 may utilize the member 14 to secure the positioning fixture 2 to the long bone 10, the positioning fixture 2 may further include a securing feature 34 cooperable with the body 12 for securing the positioning fixture 2 to the long bone 10. The securing feature 34 may have any suitable size and shape and as shown in FIG. 1, may extend inwardly from the body 12 to contact and cooperate with the long bone 10. The securing feature 34 may, for example, be in the form of a K-wire. The K-wire 34 includes a tip 36 which penetrates into the long bone 10.

While the securing feature 34 may include a solitary securing feature or K-wire 34, it should be appreciated that the positioning fixture 2 may include a plurality of securing features 34. For example, and as shown in FIG. 1, four spaced-apart securing features 34 are positioned in the body 12 and extend inwardly toward the long bone 10.

The top 36 of the K-wires 34 may extend through the inner cortical wall 35 of the long bone 10 and may interfere with the stem of the trial 4 or prosthesis 6. The K-wires 34 may thus need to be removed from the positioning fixture 2 after the members 14, 20 and 22 are installed. In this case, the K-wires 34 provide temporary securement and the members 14, 20 and 22 provide fixation during trial reduction. Further, even if the K-wires 34 do not interfere with the insertion of the trial 4 or the prosthesis 6, the K-wires 34 limit the range of motion of the trial reduction and should be removed prior to trial reduction to optimize range of motion.

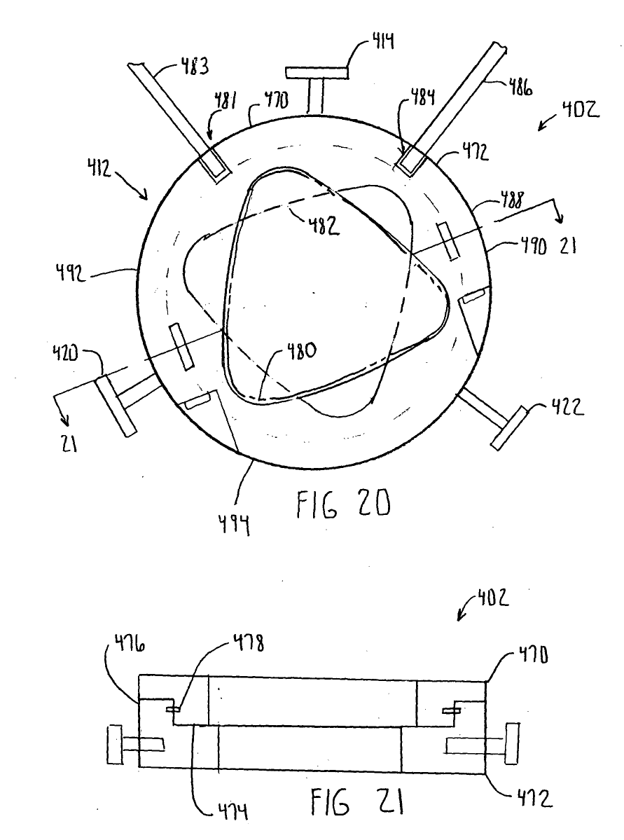

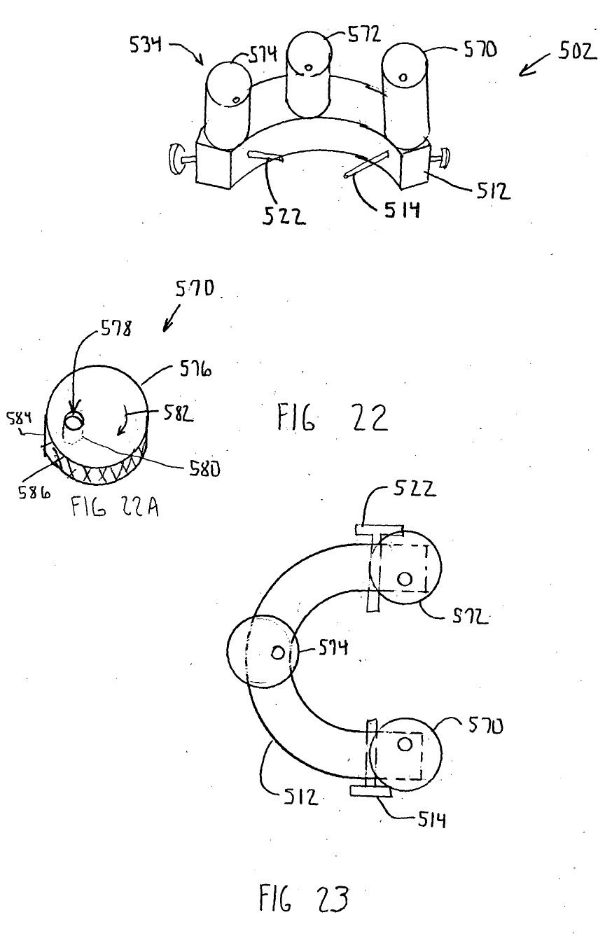

Referring now to FIG. 2, the positioning fixture 2 is shown in greater detail. In positioning feature 2 includes the body 12. The body 12 may add any suitable shape such that it may be positioned near the periphery of the long bone 10 and so that it may support and cooperate the first member 14.

While any shape of the body 12 for the positioning fixture 2 may be used in conjunction with the positioning fixture 2 of the present invention, it should be appreciated that the overall size of the body 12 should be minimized and its proximity to the periphery of the long bone 10 should be promoted so that the positioning fixture 2 may permit the reduction or the movement of the shoulder prosthesis 6 through a trial reduction that includes as close as possible the full range of motion of the shoulder joint.

To minimize this size of the positioning fixture 2, thus the positioning fixture 2 includes an inner periphery 38 which closely conforms to outer periphery 40 of the long bone 10 (see FIG. 2). Since the outer periphery 40 of the long bone 10 has a generally circular cross section, the inner periphery 38 of the positioning fixture 2 is preferably arcuate and may be defined, for example, by a radius RI extending from centerline 42. Similarly to optimize the strength while minimizing the overall size of the positioning fixture 2 includes an outer periphery 44 which is also arcuate and may be defined by a radius RO extending from outside diameter centerline 46.

To minimize the size of the positioning FIG. 2 while maximizing its strength, the positioning fixture 2 is thicker in the middle than in the outer portions of the positioning fixture 2. This varying thickness of the positioning fixture 2 may be accomplished by positioning the ID centerlines 42 and OD centerlines 46 appropriately.

The body 12 may, for simplicity, be defined by opposed spaced apart parallel faces 48. The faces 48 may be separated by a distance, for example, height H. The height H may be minimized to provide for optimum range of motion.

To maximize the allowable range of motion, the first member 14 of the positioning fixture 2, as well as, the second member 20 and the third member 22 have preferably as small a size as is practically possible. For simplicity, the head 24 of the first member 14 may have a circular shape and be defined by head diameter HD. The head 24 may extend from outer periphery 44 of the body 12 a distance HH which may be minimize to optimize the range of motion during trial reduction.

Referring now to FIG. 3, the positioning fixture 2 is shown with the members 14, 20, and 22 removed. As shown in FIG. 3, the positioning fixture 2 may include threaded openings 50 for receiving the members 14, 20, and 22. Positioning fixture 2 may also include small radial holes 52 for receiving the securing feature or K-wire 34 (see FIG. 1). While a solitary securing fixture may be used, as shown in FIG. 3, a set of four spaced apart holes 52 may be used. Each of the holes 52 may be used with a securing feature or K-wire 34.

For easily placing the positioning fixture 2 around the long bone 10 (see FIG. 1), the positioning fixture 2 defines an opening 32 which provides clearance for the long bone 10. It should be appreciated that the opening 32 may be formed by separating the first portion 16 from the second portion 18 as shown in FIG. 3. It should be further appreciated that the first portion 16 and the second portion 18 may abut each other at the opening 32 when the positioning fixture 2 is in position around the long bone 10.

Referring now to FIG. 4, positioning fixture 2 is shown with two diametrically opposed members. For example, as shown in FIG. 4, the positioning fixture 2 includes the first member 14 as well as the third member 22. As shown in FIG. 4, the body 12 of the positioning FIG. 2 may include the first portion 16 and the second portion 18. The portions 16 and 18 may be, as shown in FIG. 4, pivotally connected about body pivot centerline 54. A thumb wheel 56 may be utilized to secure and lock the first portion 16 to the second portion 18.

Referring now to FIG. 5, the first member 14 is shown in greater detail. The pin 14, as shown in FIG. 5, includes the head 24 and the stem 26 which extends to the head 24. The head 24 and the stem 26 may be in the form of cylinders and the head 24 may include a cylindrical external periphery 58. The external periphery 58 may include ribs or knurls for assisting in tightening the pin against the trial 4. The stem 26 includes an end 60 which mates with the trial 4. The end 60 may in the form of a cylindrical shape defined by stem diameter SD.

Referring now to FIG. 6, the positioning fixture 2 is shown in operation around trial 4. As shown in FIG. 6, the first portion 16 of the body 12 includes a first portion protrusion 62 which defines a first portion protrusion opening 64. Similarly, the second portion 18 includes a second portion protrusion 66 which defines a second portion protrusion opening 68.

The protrusion openings 64 and 68 receive stem 70 of the thumb screw 56. Internal threads 72 in the second portion protrusion 66 mate with external threads 74 on the stem 70 of the thumb screw 56 to secure the first portion 16 to the second portion 18. It should be appreciated that as the thumb screw 56 is loosened, the first portion 16 and the second portion 18 may pivot about pivot point 54 to permit the receiving of the long bone 10 through the opening 32 in the positioning fixture 2.

The trial 4 includes a cooperation feature 76 located on the periphery 78 of the trial 4. Preferably, as shown in FIG. 6, a plurality of spaced apart cooperation features 76 are formed on the trial 4. Cooperation features 76 may have any suitable form which permits relative motion between the positioning fixture 2 and the trial 4. The cooperation feature 76 may be in the form of a groove or slot and may extend axially, spirally, or may be in the form of a series of spaced apart openings which permit a plurality of positions between the trial 4 and the positioning fixture 2.

For simplicity and to ease in the adjustment of the trial 4 with respect to the long bone 10, the cooperation feature 76 may be in the form of an elongated void extending axially along the periphery 78 of the trial 4. Thereby the trial 4 may be moved axially when the positioning fixture 2 is in sliding contact with the trial 4. For example, as shown in FIG.6, the cooperation feature 76 may be in the form of a groove. The groove may be a V-shape groove or, as shown in FIG. 6, in the form of a groove with a generally rectangular cross section. For simplicity, the cooperation feature 76 is shown as a pair of spaced apart grooves located on opposite sides of the periphery 78 of the trial 4.

As is shown in FIG. 6, the first member 14 is engaged in first cooperation feature 82 while the third member 22 is engaged in second cooperation feature 84. When utilizing the positioning fixture 2 on the trial 4, the positioning fixture 2 is opened up with the first portion 16 separated from the second portion 18. The positioning fixture is then loaded onto the long bone 10 by inserting the positioning fixture 2 in the direction of arrow 86 until the positioning fixture 2 is in the position as shown in FIG. 6 with the first member 14 aligned with the first cooperation feature 82 and the third member 22 in alignment with the second cooperation feature 84. In order that the first member 14 may engage the first cooperation feature 82 and so that the third member 22 may engage the second cooperation feature 84, the first member 14 must be removed from the positioning fixture and a drill (not shown) is inserted through the threaded opening 50 and is used to drill a clearance hole 88 through long bone 10.

It should be appreciated that when the first member 14 is engaged with the first cooperation feature 82 and the third member 22 was in cooperation with the second cooperation feature 84, the trial 4 may be moved along trial centerline 90 of the trial 4. Thus with the positioning fixture 2 in position on the long bone 10, the height of the trial 4 or its motion along trial centerline 90, may be adjusted. The height or position along trial centerline 90 may be set at any position by tightening the first member 14 and the third member 22 against the trial 4. At any such position, a trial reduction may be performed at that position without removing the positioning fixture 2.

With the positioning fixture 2 in position on the long bone 10, as shown in FIG. 6, when the trial 4 engages the first member 14 at the first cooperation feature 82, the head centerline 92 of the trial 4 is as shown in FIG. 6. If the first member 14 and the third member 22 are retracted such that the first member 14 and the third member 22 are moved outside of the first cooperation feature 82 and outside of the second cooperation feature 84 of the trial 4, the trial 4 may be rotated in, for example, the direction of arrow 94 to obtain a more suitable version for the trial 4.

For example, if the trial 4 is rotated in the direction of arrow 94 to the position as shown in phantom, the third cooperation feature 96 will be in position with the first member 14 and the fourth cooperation feature 98 will be in alignment with the third member 22. At this position, the trial 4 will have a head centerline 100 as shown in FIG. 6. Thus the positioning feature 2 may be used to adjust the position of the head centerline from, for example, head centerline 92 to head centerline 100. Thus, the positioning fixture 2 may be utilized to adjust version within the trial 4 during a trial reduction.

Referring now to FIG. 6A, the thumb screw 56 is shown in greater detail. The thumb screw 56 includes a head 102 and a stem 70 extending from the head 102. External threads 74 are formed on the stem 70. Knurls 104 may be formed on the head 102 to assist in torquing the thumb screw 56.

Referring now to FIG. 6B, the trial 4 is shown in a first position 106 in solid. If the first member 14 is loosened on the body 12 of the positioning fixture 2, the trial 4 may be permitted to move in the direction of arrow 108 along first cooperation feature 82 to second position 110 as shown in phantom.

Referring again to FIGS. 1-6, the positioning fixture 2 may be secured to the long bone 10 in any suitable fashion. While the members 14, 20 and 22 may be utilized to secure the positioning feature 2 to the long bone 10 in addition to being used to cooperate with the trial or prosthesis, an additional feature may be utilized to fixedly secure and position the positioning fixture 2 with respect to the bone 10.

As shown in FIGS. 1-6, the securing feature 34 is in the form of a k-wire. The k-wire 34 is slidably fitted through k-wire openings 52 in the body 12. The k-wires 34 have tips 36 which penetrate the long bone 10 and after a plurality of securing features 34 are secured to the long bone 10, the positioning fixture 2 is rigidly secured to the long bone 10.

It should be appreciated that the securing feature 34 may alternatively be in the form of a series of threaded fasteners or set screws. Each of the set screws may be threadably secured to the body 12 by providing threads, not shown, on the openings 52. The fasteners or set screws may include a point or tip (not shown) to penetrate the surface of the bone 10.

Referring now to FIG. 7, a trial 4 is shown for use with the positioning fixture of FIG. 1 for positioning the trial 4 in the medullary canal 8 of the long bone 10. The trial 4 includes the cooperation feature 76.

The trial 4 is adapted to be supported at the cooperation feature 76 by the positioning fixture 2 of FIG. 1 as the trial 4 is positioned with respect to the long bone 10.

While the cooperation feature may be any feature which provides for relative motion between the trial 4 and the while the trial is being positioned within the long bone 10, the cooperation feature 76 as is shown in FIG. 7, may be in the form of a longitudinal groove. The longitudinal groove 76 as shown in FIG. 7, extends lineally in a direction parallel to longitudinal axis 112 of the trial 4. It should be appreciated that the longitudinal groove may be spiraled or have a shape other than the vertical linear shape of FIG. 7. Further it should be appreciated that the cooperation feature 76 may be in the form of a protrusion, a series of bumps, a rib, series of ribs, a depression or series of depressions.

The cooperation feature 76 may consist merely of the outer periphery of the trial. The outer periphery 76 may have a smooth contour and have an oval or round cross section. If the outer periphery has a round or circular cross section, the version of the trial may have infinite rather than discrete adjustment. Such infinite adjustment of version by merely rotating the trial to any angular position may make the use of a smooth stem with a round or oval cross section preferred. The adjustment for version may be made by providing minimal clearance between the fixture 2 and the trial 4 or by loosening the member 14, 20, and 22 prior to making a version adjustment. It should be appreciated that a plurality of members 14 may be preferred with a smooth periphery cooperating feature. In fact for such a design, three or more members 14 may be desired.

The positioning fixture 2 of FIGS. 1-6 may have any suitable size and shape capable of closely surrounding and supporting the trial 4 or the prosthesis 6 within the long bone 10. The positioning fixture 2 may be made of any suitable durable material capable of a commercial sterilization process such as an auto clave process. For example, the positioning fixture 2 may be made of a metal or a plastic. If made of a metal, the positioning fixture may be made of, for example, a cobalt chromium alloy, a stainless steel alloy or a titanium alloy.

The trial 4 of FIG. 7 may have any suitable shape capable of serving as a replication of the corresponding prosthesis. The trial 4 as shown in FIG. 7 includes a stem 114 which extends from body 116. The stem 114 fits at least partially within the medullary canal 8 of the long bone 10. A trial head 118, shown in phantom, is positioned along angular face 120 of the body 116 of the trial 4.

As shown in FIG. 7, the cooperation feature 76 is formed on periphery 122 of the stem 114. The trial 4 is utilized to be sure that the proper size prosthesis has been preliminary selected by the surgeon. Each of the available prostheses has a trial with corresponding dimensions which may be utilized in the medullary canal of the patient and positioned in the canal for the performance of a trial reduction to assure the appropriateness of the size of the trial and corresponding prosthesis and to assist in determining the proper depth and angular orientation of the trial and the respective prosthesis within the long bone 10.

The trial 4 may be made of any suitable durable material which may be sterilized by any of the available commercial techniques, including for example, auto clave. For example, the trial 4 may be made of a plastic or metal. If made of a metal, the trial 4 may be made of, for example a cobalt chromium alloy, a stainless steel alloy, or a titanium alloy.

Referring now to FIG. 8, the cooperation feature 76 of the trial 4 is shown in greater detail. The cooperation feature 76 may include a solitary feature, for example, a solitary longitudinal groove or protrusion. But preferably and as shown in FIGS. 7 and 8, the cooperation feature 76 includes a plurality of spaced apart features. By providing a plurality of spaced apart features, the cooperation feature 76 may cooperate with the positioning fixture 2 of FIG. 1 to provide for a plurality of angular orientations of the trial 4 within the long bone 10.

For example and as shown in FIG. 8, the cooperation feature 76 may include 18 equally spaced apart cooperation features 76. Each of the 18 features 76 are angularly spaced apart from each other, for example, 20 degrees. Thus, as the cooperation feature 76 is aligned with an adjoining cooperation feature to the positioning feature 2, the trial 4 may be rotated about the longitudinal axis 112 of the trial for a amount of 20, for example, degrees.

The cooperation feature 76 may have any suitable shape such that it mates with the member 14 of the positioning feature 2 (see FIG. 1). The cooperation feature 76 may be merely the smooth external periphery of the trial 4. For example, as shown in FIG. 8, the cooperation feature 76 is in the form of a longitudinal groove having a height FH and being separated from each other by an angle θ. The angle θ may be, for example, 20 degrees if there are 18 equally spaced apart longitudinal grooves 76.

As an alternative to the U shaped groove of FIG. 8, the cooperation feature may be in the form of, for example, a cooperation feature 76A of the trial 4A of FIG. BA and, for example, have a generally V shape.

Alternatively, as shown in FIG. 8B, the cooperation feature may be in the form of cooperation feature 76B on trial 4B and have a generally rectangular shape.

Referring now to FIG. 8C, the cooperation feature may be in the form of a series of spaced apart flats 76C on trial 4C.

Referring now to FIG. 8D, the cooperation feature may be in the form of a smooth external periphery 76D on stem 14D of trial 8D. The smooth external periphery 76D may have an oval, or as shown, a circular cross section. The stem 114D may be tapered or have a uniform cross section. If the periphery 76D of the cross section of stem 114D is circular and the stem 114D has a uniform cross section, ie, if the stem 114D is cylindrical, the positioning fixture 2 can be set and the trial 4D can then be rotated and raised and lowered along the longitudinal axis 112D to adjust for version and height, respectively, without any adjustments to the fixture 2. This configuration is the simplest configuration of the cooperation feature, but is the least stable configuration.

Referring now FIG. 9, the trial 4 is shown oriented such that the face 120 of the body 116 is shown in greater detail. The body 116 of the trial 4 includes an opening 126 extending from the face 120. The opening 126 receives stem 128 of the trial head 118.

Referring now to FIG. 10, the prosthesis 6 is shown in greater detail. The prosthesis 6 is for use with the positioning fixture 2 of FIGS. 1-6 for positioning the prosthesis 6 in the medullary canal 8 of the long bone 10. It should be appreciated that the positioning fixture 2 of the present invention may be utilized with either the trial 4 of FIGS. 7-9 or with the prosthesis 6 of FIGS. 10-12 or with the both the trial 4 and the prosthesis 6.

If the positioning fixture 2 is used solely with the trial, the position which is determined after trial reduction to be the proper position of the trial in the long bone 10 is recorded by any reliable method such as by scribing or determining a physical reference mark between the trial 4 and the long bone 10 or another anatomically feature. The prosthesis 6 is then positioned to that mark on anatomical feature on, for example, the long bone 10. Alternatively, the prosthesis 6 may be positioned relative to a reference mark scribed onto the long bone 10.

When the positioning feature 2 is utilized with both the trial and the prosthesis, the prosthesis 6 as shown in FIGS. 10-12, include a cooperation feature 130 which preferably is similar to the cooperation feature 76 of the trial 4. The prosthesis 6 of FIG. 10 is adapted to be supported at the cooperation feature 130 by the positioning fixture 2 of FIGS. 1-6 as the prosthesis 6 is positioned with the respect to the long bone 10.

The cooperation feature 130 of the prosthesis 6 is similar to the cooperation feature 76 of the positioning feature 2, and may have any suitable shape. The cooperation feature 130, however preferably, is similar to the cooperation feature 76 of the trial 4. The prosthesis 6 may include a cooperation feature 130 in the form of, for example, a longitudinal groove. The positioning fixture 2 may then cooperate with the cooperation feature 130. The cooperation feature 130 may, as shown in FIG. 10, be positioned on periphery 132 of stem 134. A body 136 may extend proximally from the stem 134. An angular face 138 may be formed on the body 136 for receiving the prosthetic head 140.

Referring now to FIG. 11 the cooperation feature 130 is shown in greater detail. The cooperation feature 130 as shown in FIG. 11, is in the form of a longitudinal groove. While the cooperation feature 130 may include a solitary cooperation feature as shown in FIG. 11, the cooperation feature 130 includes a plurality of spaced apart longitudinal grooves 130. For example, and as shown in FIG. 11, a total of 18 equally spaced apart longitudinal grooves 130 may be formed in the periphery 132 of the stem 134. Each of the adjacent longitudinal grooves 130 are spaced from each other and angleαα of, for example, 20 degrees. The grooves 130 may have any suitable shape to cooperate with the member 14 of the positioning fixture 2. For example, and as shown in FIG. 11, the longitudinal grooves 130 may have a U shape.

Referring now to FIG. 11A, an alternated embodiment of the present invention is shown as prosthesis 6A. Prosthesis 6A is similar to prosthesis 6 but includes longitudinal grooves 130A which have a generally V shape.

Referring now to FIG. 11B, another embodiment of the present invention is shown as prosthesis 6B. Prosthesis 6B is similar to the prosthesis 6 of FIG. 10 except that the prosthesis 6B includes longitudinal grooves 130B which have a generally rectangular cross section. Referring now to FIG. 11C, another embodiment of the present invention is shown as prosthesis 6C. Prosthesis 6C is similar to prosthesis 6 of FIG. 10 except that prosthesis 6C includes cooperation feature 130C in the form of, for example, spaced apart flats.

Referring now to FIG. 11D, another embodiment of the present invention is shown as prosthesis 6D. Prosthesis 6D is similar to prosthesis 6 of FIG. 10 except that prosthesis 6D includes cooperation feature 130D in the form of a smooth external periphery. The smooth external periphery 130D may have an oval, or as shown in FIG. 11D, a circular cross section. The stem 134D may be tapered or may have a uniform cross section, ie, if the stem 134D is cylindrical, the positioning fixture 2 can be set and the prosthesis 6D can then be rotated about axis 134D and raised and lowered along longitudinal axis 135D to set height and version, respectively, without any adjustment to the fixture 2. This configuration is the most simple of the configurations described and may be used if the fixture 2 is not used with the prosthesis 6D. If the prosthesis 6D is not used with the fixture 2, marks or bony landmarks may be used to transfer the position established by the trail 4D to the prosthesis 6D.

Referring now to FIG. 12, another view of the prosthesis 6 of the present invention shows the body 136 of the prosthesis 6 in greater detail. The body 136 of the prosthesis 6 may include a face 138 which defines an opening 142 for receiving stem 144 of the head 140 for use with the prosthesis 6 of the present invention.

It should be appreciated that the prosthesis 6 of FIGS. 10-12 and the trial 4 of FIGS. 7-9 may include an integral head, integral to the body that is similar to the head 140 and 118, respectively, which is generally convex. It should also be appreciated that the prosthesis 6 and the trial 4 may include a head (not shown) which is concave. The concave head of the prosthesis and trial would mate with a convex feature secured to the glenoid.

The prosthesis 6 may be made of any suitable durable material which is compatible with the human body and with a commercial sterilization process, for example, auto claving. The prosthesis 6 may be made of, for example, a plastic or a metal. If made of a metal, the prosthesis 6 may be made of, for example, a titanium alloy, a cobalt chromium alloy, or a stainless steel alloy.

Referring now to FIG. 13, a kit 150 for use in performing joint arthroplasty is shown. The kit 150 includes the positioning fixture 2. The kit 150 further includes the trial 4 or the prosthesis 6. The kit 150 may include both the trial 4 and the prosthesis 6. The trial 4 includes the cooperation feature 76 while the prosthesis 6 includes the cooperation feature 130.

The positioning feature 2 is utilized for positioning the trial 4 or the prosthesis 6 in the medullary canal 8 of the long bone 10. The long bone 10 may be, for example, a humerus. It should be appreciated, however, that the long bone 10 may be any long bone and particularly well suited for the humerus or the femur.

The fixture 2 includes the body 12 and the member 14 for cooperation with the body 12. The member 14 extends from the body 12 through a portion of the long bone 12 and into the medullary canal 8. The member 14 is adapted to support the trial 4 or the prosthesis 6 as the trial 4 or the prosthesis 6 is positioned with respect to the long bone 10. The member 14 may be adapted to provide relative motion between the body 12 and the trial 4 or the prosthesis 6 as the trial 4 or the prosthesis 6 cooperates with the member 14.

As shown in FIG. 13, the first member 14 may engage with a longitudinal groove 76 and the third member 22 of the positioning fixture 2 may engage with another space apart longitudinal groove 76. With the members 14 and 22 of the positioning fixture 2, in contact with the longitudinal groove 76 of the trial 4, the trial 4 may be moved along longitudinal axis 112 of the trial 4.

Referring now to FIG. 14, another embodiment of the present invention is shown as positioning fixture 202. Positioning fixture 202 is similar to the positioning fixture 2 of FIGS. 1-6 except that positioning fixture 202 includes a cap screw 270 for controlling the relative position of first portion 216 of body 212 of positioning fixture 202 with respect to second portion 218 of the body 212 of the positioning fixture 202. The cap screw 270 includes a head 272 which seats against face 274 of the second portion 218 and extends through hole 276 in the second portion 218 to engage with, for example, internal threads 278 formed opening 280 in the first portion 216. The internal threads 278 of the first portion 216 engage with external threads 282 of the screw 270.

Similar to the positioning fixture 2 of FIGS. 1-6, the positioning fixture 202 includes a body pivot centerline 254 about which the first portion 216 and the second portion 218 rotate. As the cap screw 270 is rotated in a first direction, the first portion 216 is drawn toward to the second portion 218 thereby advancing the first member 214 against the third member 222 to secure the positioning fixture 202 against, for example, the trial or the prosthesis. As the cap screw 270 is rotated in the opposite direction as previously mentioned, the first portion 216 is separated from the second portion 218. A stop 286 may be positioned against the head 272 opposite the face 274 to secure the head 272 to assist in using the cap screw 270 to separate the first portion 216 from the second portion 218.

Referring now to I 15, another embodiment of the present invention is shown as positioning fixture 302. The positioning fixture 302 is similar to the positioning fixture 2 of FIGS. 1-6 except that positioning fixture 302 is adapted to provide for angular orientation of the positioning fixture with respect to the bone.

The positioning fixture 2 of FIGS. 1-6 may be positioned angularly anywhere about the long bone. Since the positioning fixture 2 of FIGS. 1-6 may be positioned anywhere angularly about the long bone, the prosthesis 6 and the trial 4 which cooperate with the positioning fixture 2 of FIGS. 1-6 may require orientation features around the entire periphery to accommodate the lack of positioning ability of the positioning fixture 2.

The positioning fixture 302 of FIG. 15 is adapted to overcome the inability of the positioning fixture 2 of FIGS. 1-6 to provide for angular orientation. The positioning fixture 302 of FIG. 15 is similar to positioning fixture 2 of FIGS. 1-6 except the positioning fixture 302 further includes an alignment feature 370 for aligning the positioning fixture 302 to, for example, a bony landmark 372. The bony landmark 372 may be, for example, distal condyles 372 of the humerus 10. The alignment fixture 370 may be a visual orientation feature 370. The visual orientation feature 370 may be in the form of an alignment rod 370 positioned perpendicular to the line joining the condyles 372.

By positioning the positioning fixture 302 angularly about the long bone 10 such that the alignment rod 370 is perpendicular to the condyles 372, the positioning fixture 302 may be accurately angularly orientated with respect to the long bone 10.

The alignment rod 370 may be secured to the positioning fixture 302 in any suitable fashion, for example by welding, fasteners or a slip or snap fit. As shown in FIG. 15, the alignment rod 370 is matingly slip fitted to a rod locating hole 374 formed on the body 312 of the positioning fixture 302. Positioning fixture 302 may include a first member 314 extending from first position 316 of the body 312 as well as an opposed third member 322 extending from the second portion 318 of the body 312. The members 314 and 322 engage, for example, trial 304.

While the positioning fixture 302 of FIG. 15 may be utilized with the trial 4 of FIGS. 7-9 or the prosthesis 6 of FIGS. 10-12, referring now to FIGS. 16 and 17, the positioning fixture 302 is particularly well suited to trial 304. The trial 304 is similar to the trial 4 of FIGS. 7-9 except because the positioning fixture 302 is selectively angularly oriented in the proper position, the trial 304 may include a cooperation feature 376 formed on stem 314 of the trial 304 which does not extend around the entire periphery of the stem 314 but only over a portion of the stem 314.

For example, and as shown in FIGS. 16 and 17, the cooperation feature 376 may extend only a portion or sector of the periphery of the stem 314. The cooperation feature thus may extend over a angular portion β of the stem 314. The angle β may be, for example, 40 degrees to 80 degrees to accommodate the variation in version from patient to patient. The cooperation feature 376 may have any suitable shape, and may include the same shape available for the trial 2 including a u-shape, a v-shape, a rectangular shape or a series of flats.

Referring now to FIGS. 18 and 19, a prosthesis 306 is shown for use with the trial 304. Similar to the trial 304, prosthesis 306 includes a cooperation feature 330 extending from periphery 332 of stem 334 of the prosthesis 306. The feature 330 which extends over a portion of the periphery 332. The cooperation feature 330 may therefore extend an angle ββ of, for example, 40 degrees to 80 degrees to correspond to the trial 304 of FIGS. 16 and 17.

Referring now to FIGS. 20 and 21, an alternative method of physically securing the fixture of the present invention to the bone is shown as positioning fixture 402. The positioning fixture 402 is similar to the positioning fixture 2 of FIGS. 1-6 and includes a body 412 for which members, for example, members 414, second member 420 and third member 422 are slidably fitted. The members 414, 420 and 422 engage a trial or a prosthesis, for example, trial 4 or prosthesis 6.

The body 412, as shown in FIGS. 20 and 21, includes a first ring 470 and a second ring 472. The first ring 470 is rotatably fitted within the second ring 472 by lip 474 on the first ring 470, which mates with a rim 476 on the second ring 472. The lip 474 and the rim 476 may be secured into position with each other by, for example, a snap ring 478 to prevent the first ring 470 from separating from the second ring 472.

As the first ring 470 is rotated relative to the second ring 472, the first and second ring 470 and 472 engage and lock against the long bone 10. The locking by rotating of the positioning fixture 402 is accomplished by providing an internal periphery 480 of the first ring 470 and an internal periphery 482 of the second ring 472 which peripheries 480 and 482 are non-circular. The peripheries 480 and 482 may have any non-circular shape, for example, the internal peripheries 480 and 482 may be oval, rectangular, triangular, polygon or have any suitable non-circular cross-section.

For example, and as shown in FIGS. 20 and 21, internal periphery 481 and internal periphery 483 may have a first position in which the peripheries 481 and 482 are in alignment with each other and a second position in which the peripheries 480 and 482 are misaligned with each other. In this misaligned position, the positioning fixture 402 may be locked or secured against the long bone 10. The locked or misaligned position is shown in solid and the unlocked or aligned position is shown in phantom. To assist in tightening the positioning fixture 402, the first ring 470 includes a first feature 481 for cooperation with a tool 483 and the second ring 472 may include a second feature 484 for cooperating with a second tool 486 to rotate the first ring 470 with respect to the second ring 472.

For example, as shown in FIGS. 20 and 21, the first feature 481 may be in the form of a hole and second feature 484 may also be in the form of a hole. The tightening features may be in the form of, for example, a first arm 483 for cooperation with the first hole 481, and a second arm 486 for cooperation with the second hole 484. To assemble the positioning fixture 402 onto the long bone 10, the first ring 470 may be made of two portions, first portion 488 and the second portion 490. Similarly the second ring 472 may include a second ring first portion 492 and a second ring second portion 494. The portions 488 and 490 may be assembled to form the first ring 470 and the portions 492 and 494 may be assembled to form the second ring 472.

Referring now to FIGS. 22, 22A and 23, another embodiment of the positioning fixture of the present invention is shown as positioning fixture 502. Positioning 502 is similar to the positioning fixture 2 of FIGS. 1-6 except it uses a different method of securing the positioning fixture 502 to the bone 10. The positioning fixture 502 includes a body 512 similar to the body 12 of the positioning fixture 2. The body 512, may as shown in FIGS. 22 and 23, be fixed as in a generally U shape. It should be appreciated that the body 512 may alternatively pivot as the body 12 of the fixture 2 does. The body 512 includes a first member 514 for cooperation with the trial of prosthesis and may include a second or a third member 522 for cooperation with the trial or the prosthesis.

The positioning fixture 502 further includes a securing feature 534. The securing feature 534 includes a first securing feature 570. The securing feature 532 may alternatively include a spaced apart second securing feature 572 and a third securing feature 574. The securing features 570, 572, and 574 may be similar to each other. The first securing feature 570 is secured to the body 512. The securing feature 534 is in the form of a cam which may be actuated to move in the direction of the long bone 10 to secure the fixture 502 to the long bone 10.

For example, as shown in FIG. 22A the first securing fixture 570 may include a cam 576 in the form of a circular disk having a hole 578 positioned eccentric with the cam 576. A fastener 580 may be used to secure the cam 576 to the body 512 about the opening 578. To operate the cam 576, the fastener 580 is loose against the body 512 and the cam 576 is rotated in the direction of arrow 582 until periphery 584 of the cam 576 is contact and is secured against the long bone 10. The fastener 580 then is used to secure the cam 576 in the lock position. Knurls 586 may be formed on periphery 584 to assist in the securement of the fixture 502 to the long bone 10. Second securing feature 572 and 574 may, as shown in FIGS. 22 and 23, be identical to the first securing feature 570.

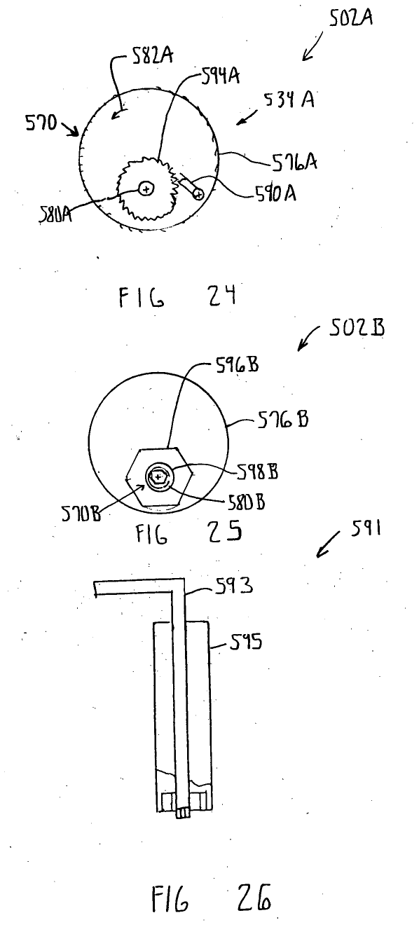

Referring now to FIG. 24, another embodiment of the present invention is shown as positioning fixture 502A. Positioning fixture 502A is similar to positioning fixture 502 but includes a securing feature 534A including first securing feature 570A. First securing feature 570A is similar to the first securing feature 570 of the positioning fixture 502 of FIG. 22 except that cam 576A of the first securing feature 570A includes a pawl 590A pivotably secured to the cam 576A. A screw 580A similar to the screw 580 secures the cam 576A to the fixture 502A. The cam 576A includes a ratchet 594A. As the cam 576A is rotated in the direction of arrow 582A, the pawl 590 engages the ratchet 594A, locking the cam 576A progressively as it moves in the direction of arrow 582A, thereby, providing a secure locking of the first securing feature 570A into the long bone 10.

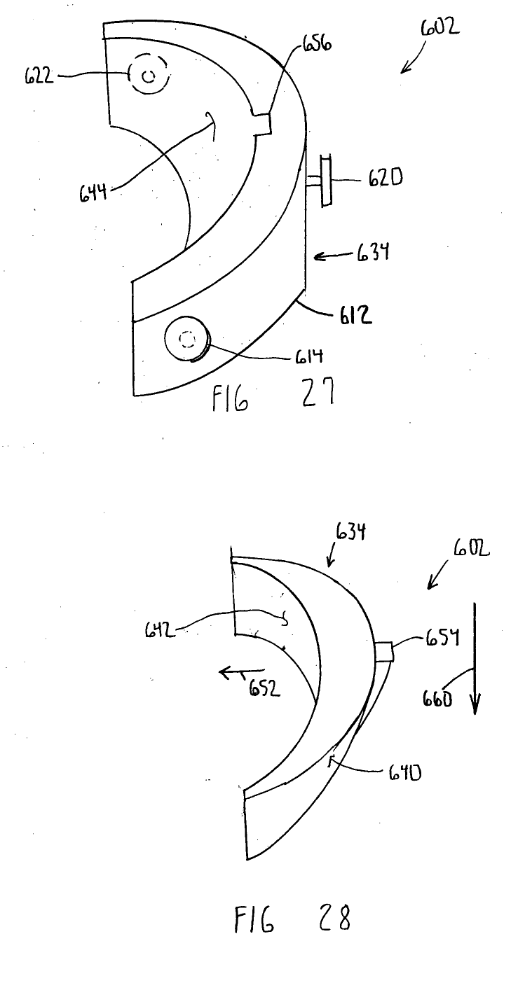

Referring now to FIG. 25, another embodiment of the present invention is shown as positioning fixture 502B. Positioning fixture 502B is similar to positioning fixture 502 of FIGS. 22 and 23 except the positioning fixture 502B includes a first securing feature 570B which includes a cam 576B including a hexagonal head 596B and a screw 580B including a socket head 5983. The hex head 5963 and the socket head 598B of the securing feature 570B of the positioning fixture 502B assist in locking securing feature 570B in a locked position.

Referring now to FIG. 26, a wrench assembly 591 is shown for use with the positioning fixture 502B of FIG. 25. The wrench assembly 591 includes an Allen wrench 593 which slideably fits within a hollow socket wrench 595.

The socket 595 is used to angularly orient the cam 576B of the positioning fixture 5023 of FIG. 25. The Allen wrench 593 is utilized to loosen and tighten the screw 580B. To utilize the wrench assembly 591 with the positioning fixture 502B of FIG. 25, the socket 595 is utilized to rotate the cam 576B in the proper position and the Allen wrench 593 is used to tightened and loosen the screw 580B.

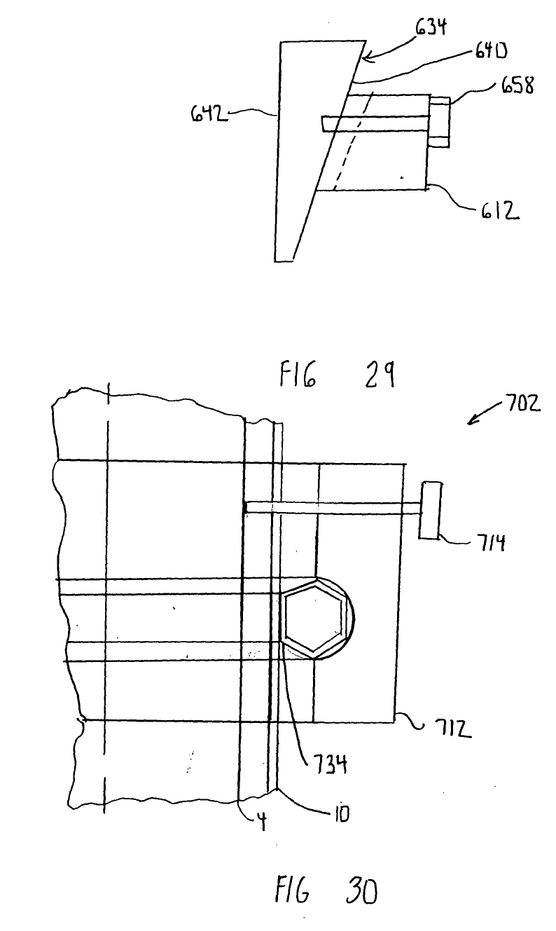

Referring now to FIGS. 27, 28 and 29, another embodiment of the present invention is shown as positioning fixture 602. Positioning 602 uses yet another method of securing the fixture to the bone. Referring now to FIG. 27, the positioning fixture 602 includes a body 612. The body 612 is used to support first member 614 for cooperation with a alignment feature of the trial or prosthesis, for example, trial 4 or prosthesis 6 (see FIGS. 7-12).

Continuing to referring to FIG. 27, the positioning fixture 602 may further include a second member 620 similar to the first member 612 as well as a third member 622 also similar to the first member 612. The first member 614 may be similar or even identical to the first member 14 of the positioning fixture 2 of FIGS. 1-6.

Referring now to FIG. 28, the securing feature 634 is in the form of a wedge. The wedge 634 includes a tapered surface 640 for cooperation with the body 612 and an opposed bone contact surface 642 for contact with the long bone 10.

Referring again to FIG. 27, the body 612 includes a body tapered surface 644 for which mates with the wedge tapered surface 640 of the wedge 634. Continuing to refer to FIGS. 27 and 28, as the wedge 634 is advanced in the direction of arrow 660, the cooperation of the wedge tapered surface 640 and the body tapered surface 644 of the body 612 urges the bone contact surface 642 of the wedge 634 in the direction of arrow 652.

Shown in FIGS. 27 and 28, a tang 654 on the wedge 634 may cooperate with a groove 656 on the body 612 to guide the wedge 634 as it moves and cooperates with the body 612. As shown in FIG. 29, fastener 658 located in the body 612 may engage with the wedge tapered surface 640 of the wedge 634 to lock the wedge 634 in engagement with the body 620.

The body 612 may be made of any suitable durable material and be made of, for example, a metal. The wedge 634 may be made of any suitable durable material and may be made with slots or grooves (not shown) to provide pliability to the wedge 634 as it is moved in engagement with the body 612. The wedge 634 may alternatively made of a pliable material, for example, a polymer. Use of a pliable material or the use of grooves in the wedge 634 will assist in providing sufficient contact between the bone contact surface 642 and the long bone 10 to assure a proper securing of the positioning fixture 602 to the long bone 10.

Referring now to FIG. 30, another embodiment of the present invention is shown as positioning fixture 702. Positioning fixture 702 is similar to positioning fixture 2 of FIGS. 1-6 but utilizes yet another method of securing the positioning fixture 702 to the long bone 10. The positioning fixture 702 includes a body 712 similar to the body 12 of the positioning fixture 2 of FIGS. 1-6. A member 714 is fitted to the body 712 for cooperation with a prosthesis or a trial, for example, trial 4 or prosthesis 6.

A securing member 734 is utilized to securely position the body 712 of the positioning fixture 702 to the long bone 10. The securing member 734 is in the form of a collapsible ring. The collapsible ring 734 may have any suitable configuration and may, for example, be in the form of a wire having, for example, a hexagonal cross section as shown FIG. 30. Alternatively, the securing member 734 may be a tube which may be filled with air or a fluid. A separate pressure device may be utilized to inflate or expand the securing member 734.

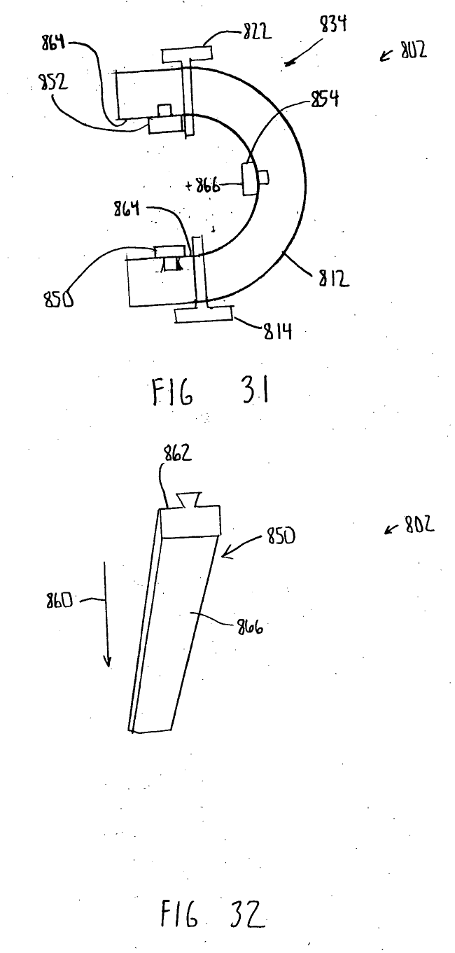

Referring now to FIGS. 31 and 32, another embodiment of the present invention is shown as positioning fixture 802. Positioning fixture 802 is similar to the positioning fixture 2 of FIGS. 1-6 but utilizes a alternate method of securing the positioning fixture 802 to the long bone 10. The positioning fixture 802 includes a body 812 similar to the body 12 of the fixture 2 of FIGS. 1-6.

A first member 814 may be used to connect the positioning fixture 802 to either a trial or a prosthesis, for example, trial 4 or prosthesis 6. Positioning fixture 802 may further include a second member 822 similar to the first member 814.

The positioning fixture 802 further includes a securing feature 834 for securing the positioning fixture 802 to the long bone 10. The securing feature 834 is somewhat similar to the securing feature 634 of the positioning fixture 602 of FIGS. 27 and 28 except that rather than the singular positioning wedge of the positioning fixture 602, the securing feature 834 of the positioning fixture 812 of FIGS. 31 and 32 includes a plurality of securing features in the form of, for example, first securing member 850 as well as a second securing member 852 and, for example, a third securing member 854. The securing members 850, 852, and 854 are shown in greater detail in FIG. 32. The first securing member 850 is in the form of a wedge. As the wedge is moved in the direction of arrow 860, the tapered surface 862 of the securing member 850 moves in connect with tapered surface 864 of the body 812 causing the bone contact surface 866 of the securing member 850 to advance toward the long bone 10, thereby, securing the body 812 of the positioning fixture 802 securely against the long bone 10.

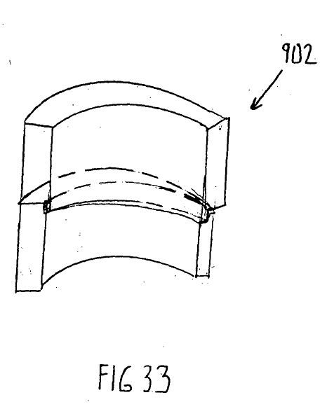

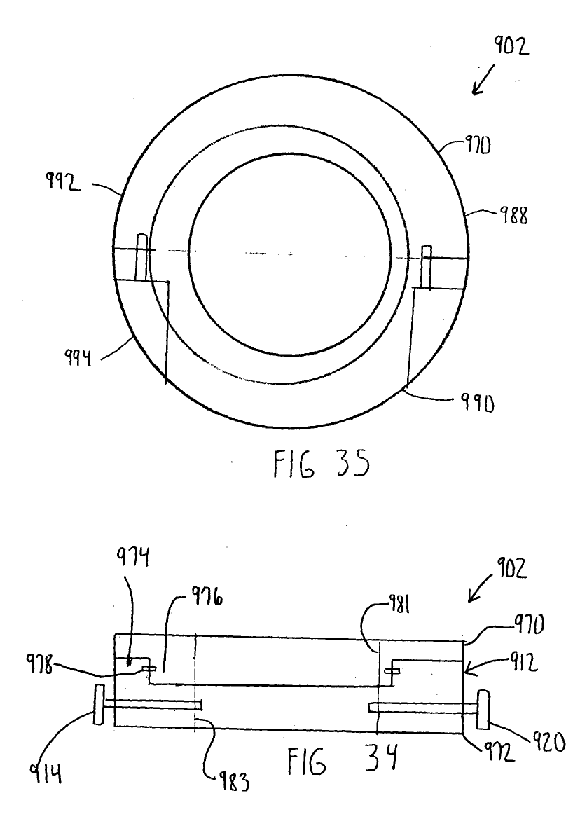

Referring now to FIGS. 33, 34, and 35, another embodiment of the present invention is shown as positioning fixture 902. Positioning fixture 902 is similar to the positioning fixture 2 of FIGS. 1-6 except the positioning fixture 902 uses a different method of securing the positioning fixture 902 against the bone 10. A positioning fixture 902 is most similar to the positioning fixture 402.

Similar to the positioning fixture 402, the positioning fixture 902 includes a first ring 970 and a second ring 972 which is rotatably secured to the first ring 970. The first ring 970 and the second ring 972 have a cylindrical outside diameter and a generally cylindrical inside diameter. The first ring 970 includes a rim 976 which cooperates with a lip 974 on the second ring 972.

The rim 976 and the lip 974 have outside diameters and inside diameters which are eccentric with each other. Thus, as the first ring 970 is rotated with respect to the second ring 972, the first ring 970 has a inner periphery 981 which in a first position is concentric with inner periphery 983 of the second ring 972. As the first ring 970 is rotated in respect to the second ring 972, the inner peripheries 981 and 983 regress from being concentric and in alignment with respect to each other to being eccentric with respect to each other. When the inner peripheries 981 and 983 are eccentric, they serve to cam lock the positioning fixture 902 against the long bone 10.

A snap ring 978 may be utilized to secure the first ring 970 to the second ring 972. To assemble the positioning fixture 902 onto the long bone 10, the first ring 970 may be made of two half circles including a first portion 988 of the first ring 970 and a second portion 990 of the first ring 970. Similarly, the second ring 972 may include a second ring first portion 992 and a second ring second portion 994.

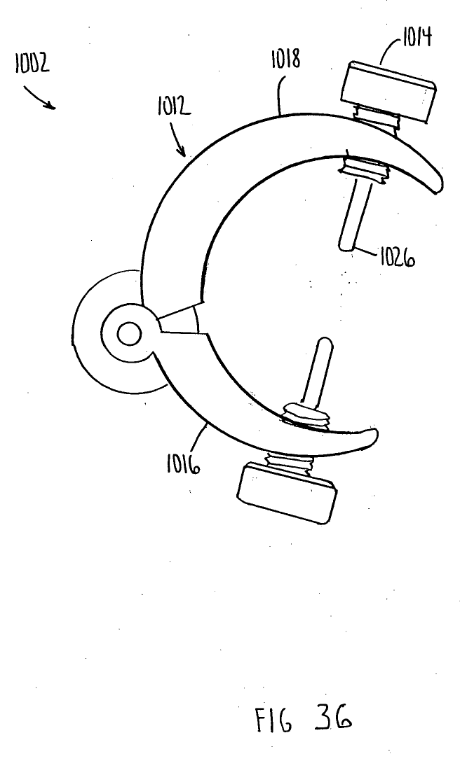

Referring now to FIG. 36, another embodiment of the present invention is shown as positioning fixture 1002. The positioning fixture 1002 is similar to the positioning fixture 2 of FIGS. 1-6 and includes a body 1012 similar to the body 12 of fixture 2. The body 1012 may include a first portion 1016 and a second portion 1018 which is pivotally secured to the first portion 1016. A member 1014 may be positioned on the body 1012 and utilized to secure the fixture 1002 to either the trial or prosthesis, for example, trial 4 or prosthesis 6.

The member 1014 is different than the member 14 of the fixture 2 of FIGS. 1-6 in that the member 1014 is configured to remain in the bone after the prosthesis 6 has been cemented and positioned in the medullary canal 8. The member 1014 may include stem 1026 which may remain in the cement after the prosthesis has been cemented and secured in position in the base.

Thus, the fixture 1012 may be utilized in conjunction with a prosthesis and be permitted to remain in position during the cementing and securing of the cement. Thus, the fixture 1002 provides for an accurate and secure positioning of the prosthesis without requiring the surgeon to physically hold the prosthesis in position during the curing process of the cement.

The stem 1026 may either be made of a material compatible with the human body, for example a metal, for example, a cobalt chromium alloy, a titanium alloy, or a stainless steel alloy. Alternatively, the stem 1026 may be made of a resorbable material. The stem 1026 may be separated from the member 1014 in any suitable fashion after the drying of the cement. For example, a rongeur may be positioned between the body 1012 and the long bone 10 and sever the stem 1026 from the member 1014.

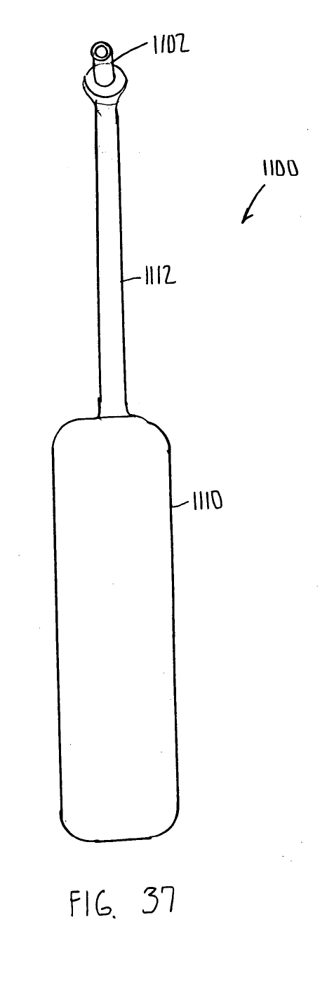

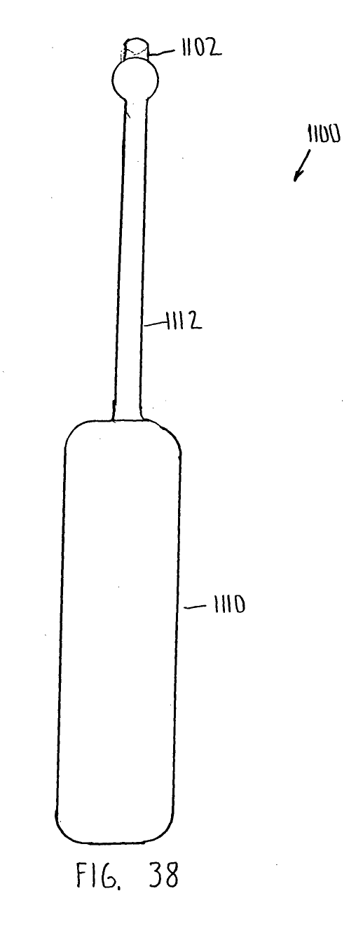

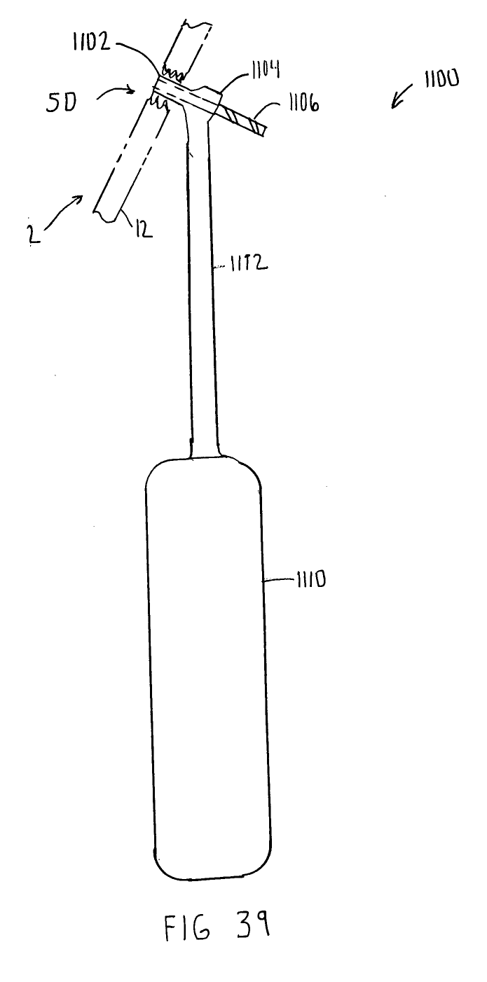

Referring now to FIGS. 37-39, a guide 1100 is shown for utilization with the positioning fixture 2 of the FIGS. 1-6. It should be appreciated that the guide 1100 may be utilized with any positioning fixture in accordance with the invention herein.

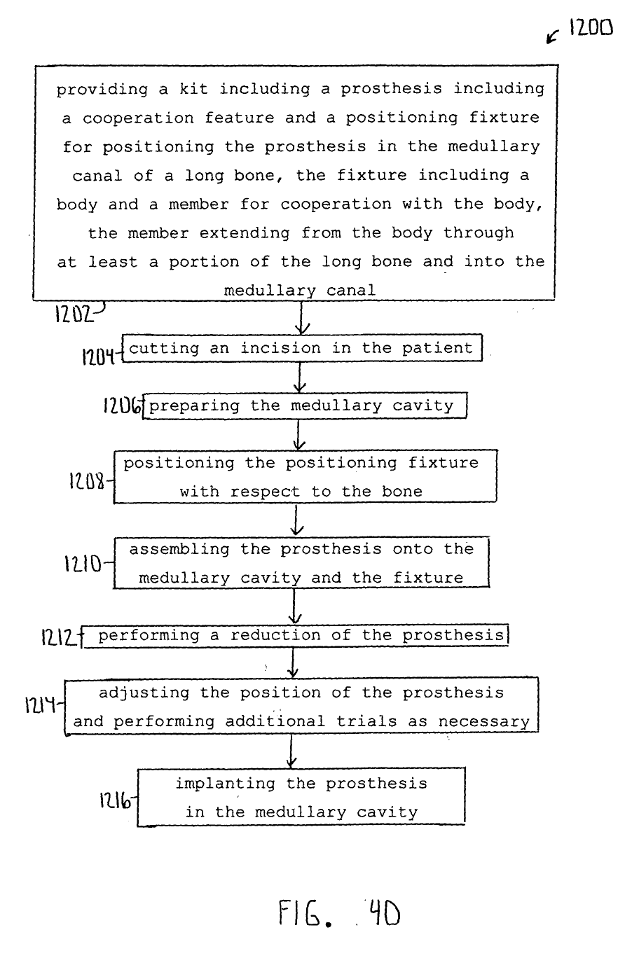

Referring now to FIG. 39, the guide 1100 is shown in position in its use in cooperation with the positioning fixture 2. The guide, 1100 includes a stem 1002 which is slideably fitted into threaded opening 50 of the body 12 of the fixture 2. Stem 1102 includes a central opening 1104 serves to guide a cutting tool, for example a drill 1106 for preparing the holes 88 in the long bone 10 for receiving the stem 26 of the pin 14 (see FIG. 6).

The guide 1100 may have any suitable shape and may, for example, include a handle 1110 for holding the guide 1100. A shaft 1112 may extend from the handle 1110. The stem 1102 extends from the shaft 1112. The guide 1100 may be made of any suitable, durable metal that is sterilizable.

Referring now to FIG. 40, another embodiment of the present invention is shown as method 1200 for providing joint arthroplasty. The method 1200 includes a first step 1202 of providing a kit include a prosthesis including a cooperation feature and positioning feature for positioning the prosthesis in the medullary canal of the long bone. The fixture includes a body and a member for cooperation of the body. The member extends from the body through at least a portion of the long bone and into the medullary canal. The method 1200 further includes a second step 1204 of cutting an incision in the patient and a third step 1206 of preparing the medullary canal. The method 1200 further includes a fourth step 1008 of positioning the positioning fixture with respect to the bone. The method 1200 further includes a fifth step 1010 of assembling the prosthesis onto the medullary canal and the fixture. The method 1200 also includes a sixth step 1212 of performing a reduction of the prosthesis and a seventh step 1214 of adjusting the position of the prosthesis and performing additional trials as necessary. The method 1200 further includes an eight step 1216 of implanting the prosthesis into the medullary canal.

Although the present invention and its advantages have been described in detail, it should be understood that various changes, substitutions, and alterations can be made therein without departing from the spirit and scope of the present invention as definite by the appended claims.

1. A positioning fixture for positioning at least one of a trial and a prosthesis in the medullary canal of a long bone, said fixture comprising:
a body; and
a member for cooperation with the body, said member extending from said body through at least a portion of the long bone and into the medullary canal, said member adapted to support at least one of the trial and the prosthesis as the one of the trial and the prosthesis is positioned with respect to the long bone.

2. The fixture of claim 1, wherein said member is adapted to permit relative motion between said body and the one of least one of the trial and the prosthesis as the one of the trial and the prosthesis cooperates with said member.

3. The fixture of claim 1, wherein said body comprises a ring for at least partially surrounding the long bone.

4. The fixture of claim 3, wherein said ring comprises:
a first portion; and
a second portion moveably attached to said first portion.

5. The fixture of claim 1, further comprising a second member spaced from said first mentioned member.

6. The fixture of claim 5, further comprising a third member spaced from said first mentioned member.

7. The fixture of claim 1, wherein said first mentioned member comprises a pin.

8. The fixture of claim 7, wherein said pin is one of threadably attached to said body and slidably attached to said body.

9. The fixture of claim 1, further comprising a securing feature cooperable with said body for securing the fixture to the long bone.

10. The fixture of claim 3, wherein said body has generally crescent shaped cross section.

11. A positioning fixture for positioning at least one of a trial and a prosthesis in the medullary canal of a long bone, said fixture comprising:
a body; and
a member for cooperation with the body, said member extending from said body through at least a portion of the long bone and into the medullary canal, said member adapted to permit relative motion between said body and the one of least one of the trial and the prosthesis as the one of the trial and the prosthesis cooperates with said member.

12. The fixture of claim 11, wherein said member is adapted to support at least one of the trial and the prosthesis as the one of the trial and the prosthesis is positioned with respect to the long bone.

13. The fixture of claim 11, wherein said body comprises a ring for at least partially surrounding the long bone.

14. The fixture of claim 13, wherein said ring comprises:
a first portion; and
a second portion moveably attached to said first portion.

15. The fixture of claim 11, further comprising a second member spaced from said first mentioned member.

16. The fixture of claim 11, wherein said first mentioned member comprises a pin.

17. The fixture of claim 16, wherein said pin is one of threadably attached to said body and slidably attached to said body.

18. The fixture of claim 11, further comprising a second member spaced from said first mentioned member.

19. The fixture of claim 11, further comprising a securing feature cooperable with said body for securing the fixture to the long bone.

20. The fixture of claim 13, wherein said body has generally crescent shaped cross section.

21. A kit for performing joint arthroplasty, said kit comprising:
one of a trial and a prosthesis, said one of said trial and said prosthesis including a cooperation feature; and
a positioning fixture for positioning at least one of a trial and a prosthesis in the medullary canal of a long bone, said fixture including a body and a member for cooperation with the body, the member extending from the body through at least a portion of the long bone and into the medullary canal, the member adapted to support at least one of the trial and the prosthesis as the one of the trial and the prosthesis is positioned with respect to the long bone.

22. The kit of claim 21, wherein the cooperation feature comprises at least a portion of the external periphery of said one of said trail and said prosthesis.

23. The kit of claim 22, wherein the external periphery has a generally circular cross section.

24. The kit of claim 21, wherein said member is adapted to permit relative motion between the body and the one of least one of the trial and the prosthesis as the one of the trial and the prosthesis cooperates with said member

25. The kit of claim 21, wherein said body comprises a ring for at least partially surrounding the long bone.

26. The kit of claim 25:
wherein said ring defines an opening therethrough;
wherein said member is moveably positional within the opening; and
wherein the cooperation feature includes a longitudinal rib which is adapted to cooperate with said member.

27. The kit of claim 21, wherein said member comprises a plurality of generally equally spaced apart members.

28. The kit of claim 21, wherein said member comprises a pin.

29. The kit of claim 28, wherein said pin is slidably fitted to the cooperation feature.

30. The kit of claim 21, further comprising the other of said trial and said prosthesis.

31. The kit of claim 21, further comprising a securing feature cooperable with said body for securing the fixture to the long bone.

32. The kit of claim 25, wherein the body of said fixture has generally crescent shaped cross section.

33. A prosthesis for use with a positioning fixture for positioning said prosthesis in the medullary canal of a long bone, said prosthesis comprising a cooperation feature, said prosthesis being adapted to be supported at the cooperation feature by the positioning fixture as the prosthesis is positioned with respect to the long bone.

34. The prosthesis of claim 33, wherein said prosthesis defines a longitudinal groove therein, the positioning fixture cooperating with said prosthesis at the groove.

35. The prosthesis of claim 33, wherein the cooperation feature comprises at least a portion of the external periphery of said prosthesis.

36. The prosthesis of claim 35, wherein the external periphery has a generally circular cross section.

37. A trial for use with a positioning fixture for positioning said trial in the medullary canal of a long bone, said trial comprising a cooperation feature, said trial being adapted to be supported at the cooperation feature by the positioning fixture as the trial is positioned with respect to the long bone.

38. The trial of claim 37, wherein said trial defines a longitudinal groove therein, the positioning fixture cooperating with said trial at the groove.

39. The trial of claim 37, wherein the cooperation feature comprising at least a portion of the external periphery of said trial.

40. The trial of claim 39, wherein the external periphery has a generally circular cross section.

41. A method for providing joint arthroplasty comprising:
providing a kit including a prosthesis including a cooperation feature and a positioning fixture for positioning the prosthesis in the medullary canal of a long bone, the fixture including a body and a member for cooperation with the body, the member extending from the body through at least a portion of the long bone and into the medullary canal;
cutting an incision in the patient;
preparing the medullary cavity;
positioning the positioning fixture with respect to the bone;
assembling the prosthesis onto the medullary cavity and the fixture;
performing a reduction of the prosthesis;
adjusting the position of the prosthesis and performing additional trials as necessary; and
implanting the prosthesis in the medullary cavity.
